(19) United States
(12) Patent Application Publication
(10) Pub. No.: US 2024/0355483 A1
(43) Pub. Date: Oct. 24, 2024

(54) PATIENT PATHWAY RECONSTRUCTION AND AGGREGATION

(71) Applicant: Roche Molecular Systems, Inc., Pleasanton, CA (US)

(72) Inventors: Charles ALCORN, Pleasanton, CA (US); Celia BEL, Basel (CH); Fernando GARCIA-ALCALDE, Basel (CH); Marie Elisabeth Stobbe KAMMERSGAARD, Meggen (CH); Enrique VIDAL OCABO, Basel (CH); Ju ZHANG, Santa Clara, CA (US); Daniel GARELLICK, Basel (CH); Carsten MAGNUS, Zurich (CH)

(21) Appl. No.: 18/560,311

(22) PCT Filed: May 11, 2022
(86) PCT No.: PCT/US2022/028749

§ 371 Date: Nov. 10, 2023

Related U.S. Application Data
(60) Provisional application No. 63/187,211, filed on May 11, 2021.

(51) Int. Cl. G16H 50/70; G16B 45/00; G16H 10/60; G16H 50/30

(52) U.S. Cl. CPC G16H 50/70; G16B 45/00; G16H 10/60; G16H 50/30

(57) ABSTRACT

Techniques for patient data management include obtaining medical history data of patients from a database, the medical history data including a history of one or more diagnosis events, one or more treatment events, and a clinical outcome event for each patient of the patients. For each patient, based on the medical history data, a computing system generates a patient pathway that includes a graph including nodes of one or more diagnosis events, the one or more treatment events, and the clinical outcome event. The computing system receives, via an interface, one or more criteria to select a subset of the patient pathways of the patients. The computing system selects, based on the one or more criteria, graphs representing the subset of the patient pathways. The computing system aggregates the subset of the patient pathways into a merged graph. The computing system displays the merged graph in the interface.

### CROSS-REFERENCE TO RELATED APPLICATIONS

This application claims priority to U.S. Provisional Application No. 63/187,211, filed on May 11, 2021, the contents of which is incorporated by reference herein in its entirety.

### BACKGROUND

Medical data management systems store information associated with events and records of a patient. These records may reflect a sequence of diagnosis, treatment, and/or the medical outcome of a patient. Such records can provide information about why, when, where, and how the patient accesses healthcare.

A clinical guideline generally refers to a document with the aim of guiding decisions and criteria regarding diagnosis, management, and treatment in specific areas of healthcare. The clinical guideline may provide the most current data about prevention, diagnosis, prognosis, therapy including dosage of medications, risk/benefit and cost-effectiveness of a treatment for a particular disease. The guideline may also identify all available (or known) decision/treatment options at a particular stage of the disease and their possible outcomes. Based on a current medical condition of a patient, a clinician can refer to the guideline to obtain the different treatment options and possible outcomes, and can determine a treatment option for the patient.

While a clinical guideline can provide bases for prescribing a particular treatment for a patient, it typically does not provide insights into how to improve the patient care. For example, while a clinical guideline can provide different treatment options and possible outcomes, it does not provide insight into real-world patient outcomes.

### BRIEF SUMMARY

Disclosed herein are techniques for reconstructing and aggregating patient pathways of a plurality of patients, based on medical history data of the patients. For each patient, the medical history data can include a historical sequence of a diagnostic event in which the patient is first diagnosed of a disease, one or more treatment events following the diagnostic event, and a clinical outcome event with respect to time. The diagnostic event can include, for example, biomarker testing events, lab testing events, etc. Moreover, the historical sequence may also include follow-up testing events after the one or more treatments. A patient pathway reconstruction and aggregation system can obtain the medical history data of the patients from a database, and generate a graph representing a patient pathway for each of the patients. The graph can include a plurality of nodes, including a start node, an end node, and nodes representing a diagnostic event, one or more treatment events, and a clinical outcome event between the start node and the end node, with the nodes connected by edges.

The patient pathway reconstruction and aggregation system can also receive, via an interface, an input to select and to display a subset of the patient pathways of the patients in the interface. The input also includes one or more criteria to select the subset of the patient pathways. Based on the input, the patient pathway reconstruction and aggregation system can select a subset of the graphs representing the subset of the patient pathways, and merge the subset of graphs into a merged graph. The input can be received via a graphical user interface.

The system can select the subset of the graphs to create the merged graph based on various criteria received as part of the instruction. For example, the criteria may also specify a number (e.g., four, eight, ten, etc.) of the most common patient pathways shared by the patients. The system can then merge the number of the most common patient pathways into a merged graph. As another example, the input may include criteria for selecting one or more common treatment events, such as a treatment event shared by a threshold number/percentage of the patients. The system can then select the subset of patient pathways having the common treatment event(s) that satisfy the criteria. As another example, the system can select a subset of the graphs based on a cluster of patients with similar features. As another example, the system can merge events into higher level categories.

In some examples, the system can generate additional analytic data of the subset of patient pathways to provide clinical and operational insights into the clinical care received by the patients. For example, the system can generate various metrics for each of the subset of patient pathways, and display the metrics in the interface. As another example, the system can retrieve medical history data of a subset of the patients who share a particular treatment event. Additional analyses, such as cumulative survival probability, a clinical outcome prediction, a next treatment event prediction, etc., can be performed based on the medical history data of the subset of the patient, and the results of the analysis can also be displayed in the interface. In some examples, discriminating treatment event nodes associated with mutually exclusive subsets of patients can be identified and selected. The medical history data of the mutually exclusive subsets of patients can also be accessed and compared based on selection of the nodes.

These and other exemplary embodiments are described in detail below. For example, other embodiments are directed to systems, devices, and computer readable media associated with methods described herein.

A better understanding of the nature and advantages of examples of the present disclosure may be gained with reference to the following detailed description and the accompanying drawings.

### BRIEF DESCRIPTION OF THE DRAWINGS

The detailed description is set forth with reference to the accompanying figures.

FIG. 1A illustrates an example of a patient pathway.

FIG. 1B illustrates examples of a clinical guideline.

FIG. 2 illustrates an example of a pathway reconstruction and aggregation system, according to certain aspects of this disclosure.

FIG. 3A, FIG. 3B, FIG. 3C, FIG. 3D, FIG. 3E, and FIG. 3F illustrate examples of operations of internal components of the pathway reconstruction and aggregation system of FIG. 2, according to certain aspects of this disclosure.

FIG. 4A and FIG. 4B illustrate examples of operations of internal components of pathway reconstruction and aggregation system of FIG. 2, according to certain aspects of this disclosure.

FIG. 5A and FIG. 5B illustrate examples of operations of internal components of the pathway reconstruction and aggregation system of FIG. 2, according to certain aspects of this disclosure.

FIG. 6A, FIG. 6B, FIG. 6C, FIG. 6D, FIG. 6E, FIG. 6F, and FIG. 6G illustrate examples of operations of a graphical user interface of pathway reconstruction and aggregation system of FIG. 2, according to certain aspects of this disclosure.

FIG. 7 illustrates an example of a method of generating and displaying patient pathways, according to certain aspects of this disclosure.

FIG. 8 illustrates an example computer system that may be utilized to implement techniques disclosed herein.

### DETAILED DESCRIPTION

A patient pathway can refer to a sequence of diagnosis, treatment, and medical outcome of a patient. The patient pathway can provide information about why, when, where, and how a patient accesses healthcare. The patient pathway of a patient can be developed based on one or more treatments selected for the patient, which can determine the clinical outcome (e.g., survival or death) of the patient. The selection of a treatment for a patient is typically based on the patient's medical conditions and clinical guidelines for treating the patient's medical conditions.

A clinical guideline typically provides the most current data about prevention, diagnosis, prognosis, therapy including dosage of medications, risk/benefit and cost-effectiveness of a treatment for a particular disease. The guideline may also identify all available (or known) decision/treatment options and their outcomes at a particular stage of the disease or the treatment, and different options/outcomes at each can be identified at different stages of the disease/treatment. Based on a current medical condition of a patient, a clinician can refer to the guideline to obtain the different treatment options and possible outcomes, and can determine a treatment option for the patient. Examples of a clinical guideline may include National Comprehensive Cancer Network® (NCCN) Clinical Practical Guidelines in Oncology.

While a clinical guideline can provide bases for prescribing a particular treatment for a patient, its usefulness in improving the clinical care provided to a patient can be limited for several reasons. Specifically, while a clinical guideline may list a number of treatment options and their outcomes, the guideline typically does not provide insight into which treatment option is the best for a particular patient. For example, the guideline typically does not provide comparison in various metrics such as survival rate, cost, duration, etc. among different treatment and/or surgery options to enable a clinician to select the best treatment/surgery regime for the patient. Moreover, the guideline also does not provide information to gain clinical and operational insights. For example, a clinician cannot obtain clinical insights, such as identifying unmet needs in clinical care, potential deviations in clinical care, etc., from the clinical guideline. Moreover, a clinician also cannot obtain operational insights, such as identifying clinical workflow inefficiency, resource management optimization opportunities, etc., from the clinical guideline.

Disclosed herein are techniques for reconstructing and aggregating patient pathways of a plurality of patients, based on medical history data of the patients. For each patient, the medical history data can include a historical sequence of (1) diagnostic events (also referred to as a diagnosis event), where a diagnostic event corresponds to diagnosing a patient with a disease, (2) one or more treatment events following the diagnostic event, and (3) a clinical outcome event. These events can be stored with respect to time (e.g., timestamped). A treatment event can include, for example, a therapy regimen, a surgery regiment, etc., each of which can include administering one or more doses of medications and/or surgery operations over a period of time. The clinical outcome event can include, for example, survival or death at a pre-determined time from the diagnosis, which can be based on a specific study period and adjusted for year of diagnosis.

Specifically, a patient pathway reconstruction and aggregation system can obtain the medical history data of the patients from a database, and generate a graph representing a patient pathway for each of the patients. The graph can include a plurality of nodes, including a start node, an end node, and nodes representing a diagnostic event, one or more treatment events, and a clinical outcome event between the start node and the end node, with the nodes connected by edges. The graph can also represent the temporal relationships among the different events, with the positions of the nodes and the directions of the edges being based on the timing of the events reflected in the medical history data. In some examples, the patient pathway reconstruction and aggregation system can traverse through a medical history record of a patient that arranges the events following a temporal order, create a node for each event, and add the nodes to the graph following the temporal order.

The patient pathway reconstruction and aggregation system can also receive, via an interface, an input to select a subset of the patient pathways of the patients. The input can include one or more criteria to select the subset of the patient pathways. Based on the input, the patient pathway reconstruction and aggregation system can select a subset of the graphs representing the subset of the patient pathways, and merge the subset of graphs into a merged graph. The merging can include merging nodes of different graphs representing a same type of diagnosis event, a same type of treatment event, or a same type of clinical outcome event into a merged nodes, and based on merging the edges between two merged nodes into a single edge. There is useful information to be obtained from looking at patient pathways at a population level (e.g., trends in patient outcome, cost, and treatment time of different pathways). However, generally, such visualizations are not useful due to the large amount of disparate patient data involved, resulting in a messy appearance from which it is difficult to discern meaning. By merging the nodes representing the same types of events, the graph is simplified and a clinician can discern information useful in improving treatment for patients.

Specifically, the system can determine that two nodes of two diagnosis events of two patients represent the same type of diagnosis event if the two patients have the same diagnosis (e.g., the same type of cancer and at the same stage), and merge the two nodes into a merged node representing the same type of diagnosis event. Moreover, the system can determine that two nodes of two treatment events of two patients represent the same type of treatment event if the two events have the same therapy/surgery regimes, and merge the two nodes into a merged node representing the same treatment event. Further, the system can also merge two nodes of identical types of clinical outcome events (e.g., survival or death), and merge the two nodes into a merged node representing the same clinical outcome event. In all these cases, two nodes can be merged whether or not the events represented by the nodes occur within the same or different time periods. In some examples, the merging can also include clustering a pair of nodes representing different diagnosis/treatment events that are close in time and/or connected by a large number of edges, or nodes that are selected as part of the input via the graphical user interface, into a single node.

The system can select the subset of the graphs to create the merged graph based on various criteria received as part of the instruction. For example, the criteria may specify that all graphs, or all patients, are included in the merged graph. The criteria may specify that only a percentage of patients (e.g., 100%, 70%, 50%, 10%, etc.) are to be represented in the merged graph, and the system can select the subset of the graphs representing the specified percentage of patients and include them in the merged graph. As another example, the criteria may also specify a number (e.g., four, eight, ten, etc.) of the most common patient pathways shared by the patients. The system can then merge the number of the most common patient pathways into a merged graph.

In some examples, the input may include criteria for selecting one or more common treatment events, and the system can select the subset of patient pathways having the one or more common treatment events. For example, the criteria may specify a specific treatment regime or a specific surgery regime, and the system can select a patient pathway having a treatment event node of the specific treatment regime or the specific surgery regime as part of the subset of patient pathways. As another example, the criteria may specify that a common treatment event shared by a threshold percentage of patients, or a threshold percentage of patients transition from a first common treatment event to a second common treatment event, and the system can select the subset of patient pathways having the common treatment event(s) that satisfy the criteria.

In some examples, the system can generate additional analytics data of the subset of patient pathways, which can provide clinical insights and operational insights into the clinical care received by the patients. For example, the system can generate various metrics (e.g., total time, total cost, total number of patients, survival rate of the patients, deviation (if any) from a clinical guideline, etc.) for each patient pathway and/or for each event node represented in the merged graph, and display the metrics in the interface. The metrics can be overlaid on the merged graph and/or displayed in a separate graph. The displaying of the metrics enables a comparison between the patient pathways to gain clinical and operational insights. For example, through comparing the survival rates of the patient pathways, a particular treatment/surgical regime can be identified to improve the survival rates. Moreover, if the patient pathways show that different treatments are prescribed to patients having the same medical conditions, potential deviations in clinical care from clinical guidelines can also be identified. Further, by comparing the total cost and/or total time of patient pathways, potential inefficiencies in clinical workflow and resource management can also be identified.

In addition, the system can also support the identification and analysis of different patient cohorts. For example, each node of a common event in the merged graph can be associated with a cohort of patients, and the medical history data of the subset of the patients can be accessed from the database via a selection of the node at the interface. Various analyses, such as the cumulative survival probability with respect to time to generate a Kaplan-Meier (K-M) plot, can then be performed on the medical history data of the cohort of the patients. In some examples, discriminating treatment event nodes associated with mutually exclusive cohorts of patients can be identified and selected. The medical history data of the mutually exclusive cohorts of patients can also be accessed and compared based on selection of the nodes. The system may further perform a prediction based on the analysis results of the cohort of the patients. For example, the system may predict a clinical outcome or a next treatment step of a patient having a treatment event in the merged graph but whose patient journey has not yet reached the end, based on a similarity between the patient and the cohort of patients associated with the treatment event.

With the disclosed techniques, a system can reconstruct patient pathways from the medical history data of patients, and display the patient pathways in a merged graph together with various metrics in an interface. The system can also selectively display a subset of the patient pathways to a user based on the user's input, to provide visualization of patient pathways of interest to the user. The system can also support exploratory analytics and hypotheses creation through displaying the metrics data (e.g., time, cost, outcomes, etc.) of the pathways, which can provide clinical and operational insights into the clinical treatments received by the patients. All these can improve the clinical care provided to future patients.

### I. Examples of Patient Pathway Development

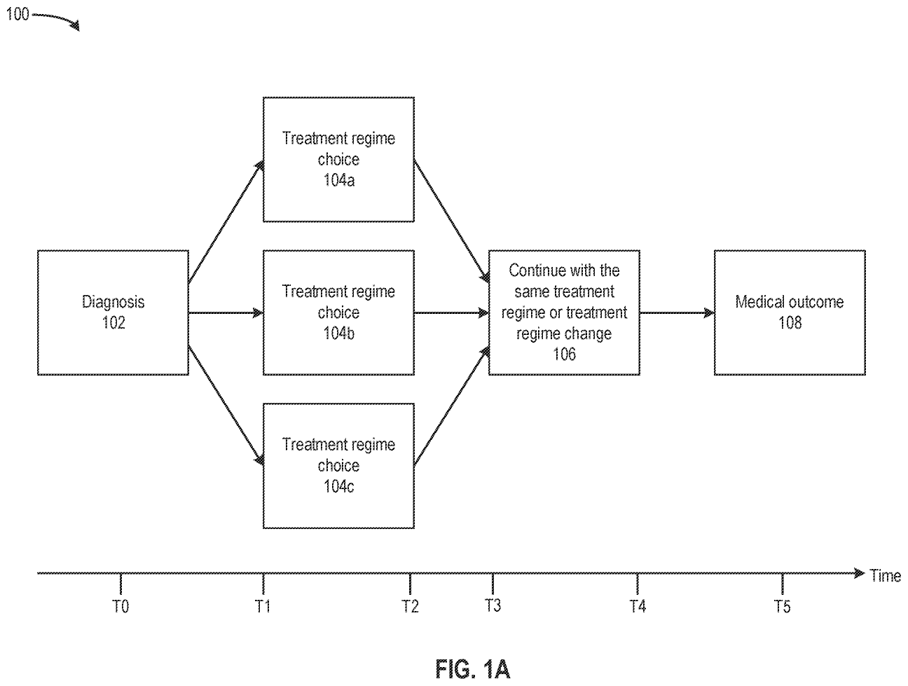

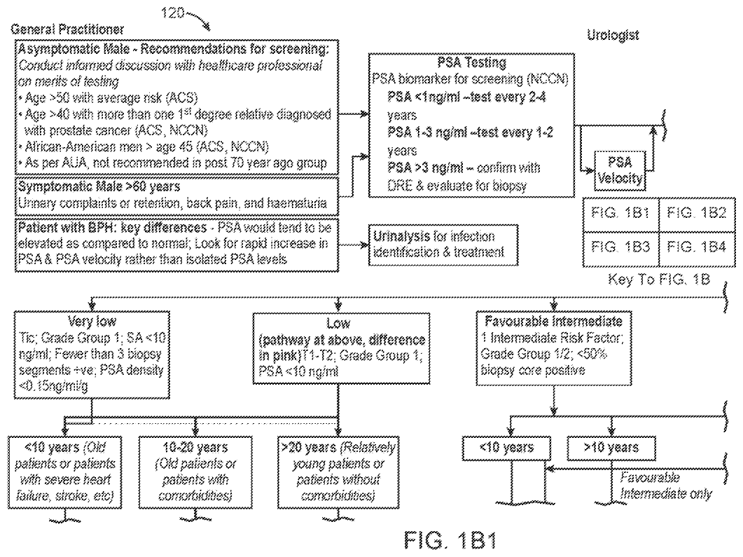

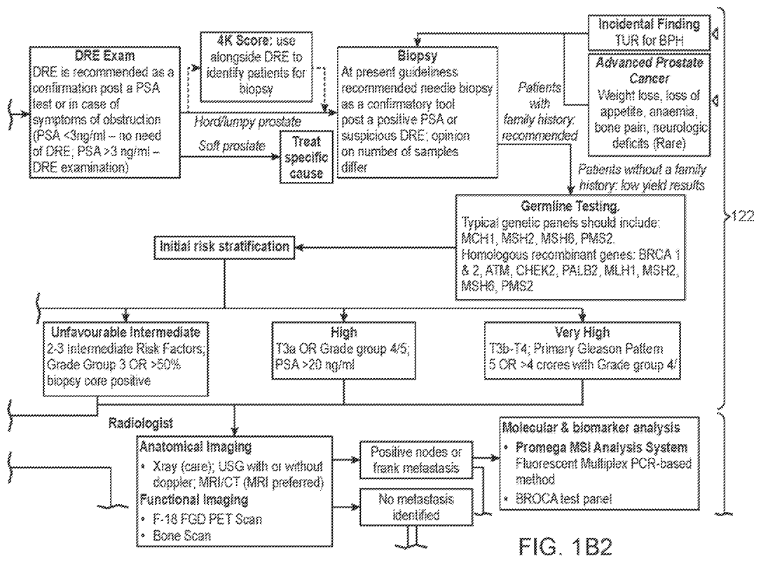

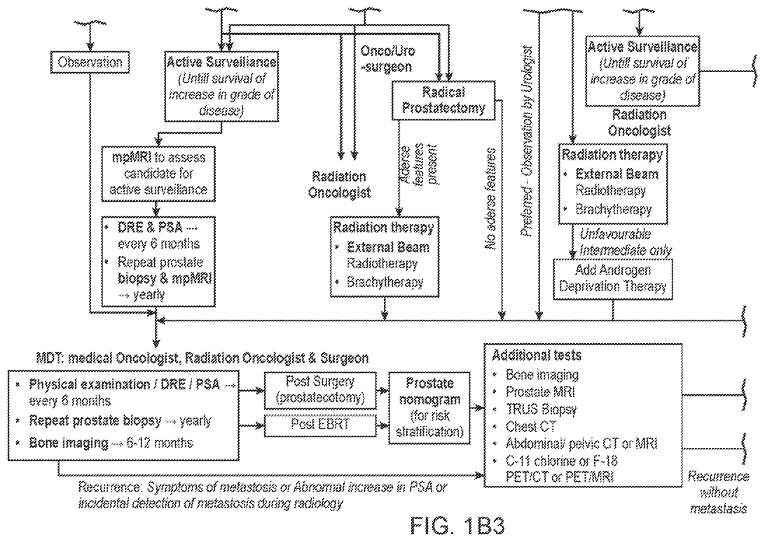

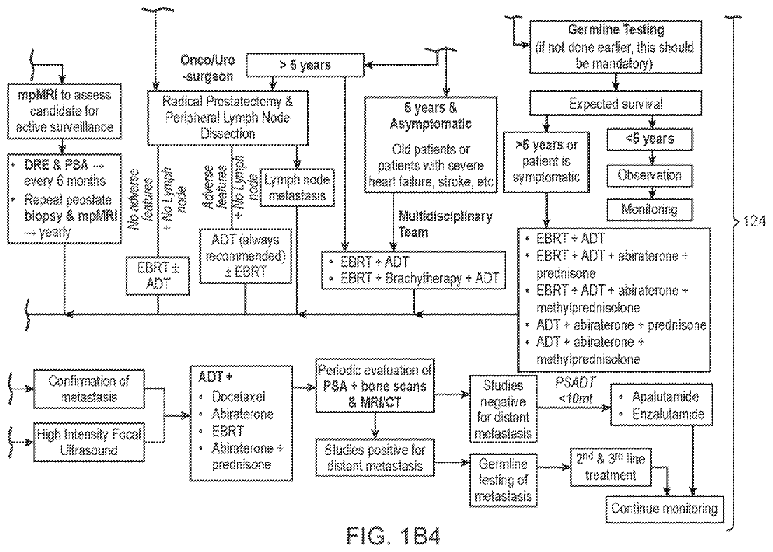

FIG. 1A and FIG. 1B illustrate examples of a patient pathway 100 and a clinical guideline 120. A patient pathway can refer to a sequence of diagnosis, treatment, and the medical outcome of a patient. Patient pathways can provide information about why, when, where, and how the patient accesses healthcare. A clinical guideline generally refers to a document with the aim of guiding decisions and criteria regarding diagnosis, management, and treatment in specific areas of healthcare. The clinical guideline may provide the most current data about prevention, diagnosis, prognosis, therapy including dosage of medications, risk/benefit and cost-effectiveness of a treatment for a particular disease. Patient pathways and clinical guidelines can both be used by a clinician to assess a patient's treatment history, different treatment options and possible outcomes, which can be used to determine a treatment option for the patient.

### A. Patient Pathway Example

FIG. 1A illustrates an example of a patient pathway 100. A patient pathway can refer to a sequence of diagnosis, treatment, and the clinical outcome of a patient, with respect to time. As shown in FIG. 1A, patient pathway 100 can start with a disease diagnosis 102 by a clinician at time T0. Based on the result of disease diagnosis 102, a treatment regime decision, from one of treatment choices 104a, 104b, or 104c, can be made by the clinician, and the patient can undertake the treatment regime between times T1 and T2. Each treatment choice can include a therapy (e.g., prescription of certain medications), a surgical operation, or both. Optionally, the medical condition of the patient after the treatment can be analyzed, and the patient can continue with the treatment regime or can receive a different treatment regime, in step 106 between times T3 and T4. After the treatment regime completes, a medical outcome 108 of the patient can occur at time T5. The medical outcome can include, for example, survival or death of the patient within a certain time (e.g., 1 year) after the treatment completes.

The selection of a treatment regime for a patient, which can decide the rest of the patient journey of the patient, is typically based on preconceived perceptions of how the patient journey of the patient should be, and is often predicated on the patient's medical conditions and clinical guidelines. The clinical guideline may provide the most current data about prevention, diagnosis, prognosis, therapy including dosage of medications, risk/benefit and cost-effectiveness of a treatment for a particular disease. The guideline may also identify all available (or known) decision/treatment options at a particular stage of the disease and their possible outcomes. Based on a current medical condition of a patient, a clinician can refer to the guideline to obtain the different treatment options and possible outcomes, and can determine a treatment option for the patient.

### B. Clinical Guideline Example

FIG. 1B illustrates an example of a clinical guideline 120 for prostate cancer treatment, which can be adapted from National Comprehensive Cancer Network® (NCCN) Clinical Practical Guidelines in Oncology and Acute Coronary Syndromes (ACS) Guidelines. As shown in FIG. 1B, clinical guideline 120 for prostate cancer treatment can include a diagnosis section 122 and a treatment section 124. Diagnosis section 122 can include various exams and biopsy operations performed by general practitioners and urologists, whereas treatment section 124 includes multiple alternative treatment options, such as radiation therapy, prostatectomy, androgen deprivation therapy, etc. A treatment option of treatment section 124 can be selected based on a risk assessment from diagnosis section 122.

As described above, while clinical guideline 120 can provide bases for prescribing a particular treatment for a prostate cancer patient, its usefulness in improving the clinical care provided to the patient can be limited. Specifically, while clinical guideline 120 lists a number of treatment options, it does not provide insight into which treatment option is the best for a particular patient. For example, clinical guideline 120 does not provide comparison in various metrics such as survival rate, cost, duration, etc. among different treatment and/or surgery options to enable a clinician to select the best treatment/surgery regime for the patient. Moreover, clinical guideline 120 also does not provide information to gain clinical and operational insights. For example, a clinician cannot obtain clinical insights, such as identifying unmet needs in clinical care for a prostate cancer patient and potential deviations in clinical care for prostate cancer patient from clinical guideline 120. Moreover, a clinician also cannot obtain operational insights, such as identifying clinical workflow inefficiency, resource management optimization opportunities, etc., from clinical guideline 120. Such insights can be obtained from analyzing patient pathways undertaken by a large group of patients, as described herein.

### II. Patient Pathway Reconstruction and Aggregation

A patient pathway reconstruction and aggregation system can produce aggregated patient pathways that show the journey of multiple patients in a user-friendly fashion. As noted above, individual patient pathways and clinical guidelines are useful in evaluating treatment options for a patient. But, the individual pathway and clinical guidelines do not show how patients respond at the population level. The patient pathway reconstruction and aggregation system solves these issues and others by generating streamlined aggregated patient pathways that a clinician can interact with to drill down into various levels of useful data and analytics, which helps the clinician to provide a better patient experience and better outcomes.

### A. Patient Pathway Reconstruction and Aggregation System

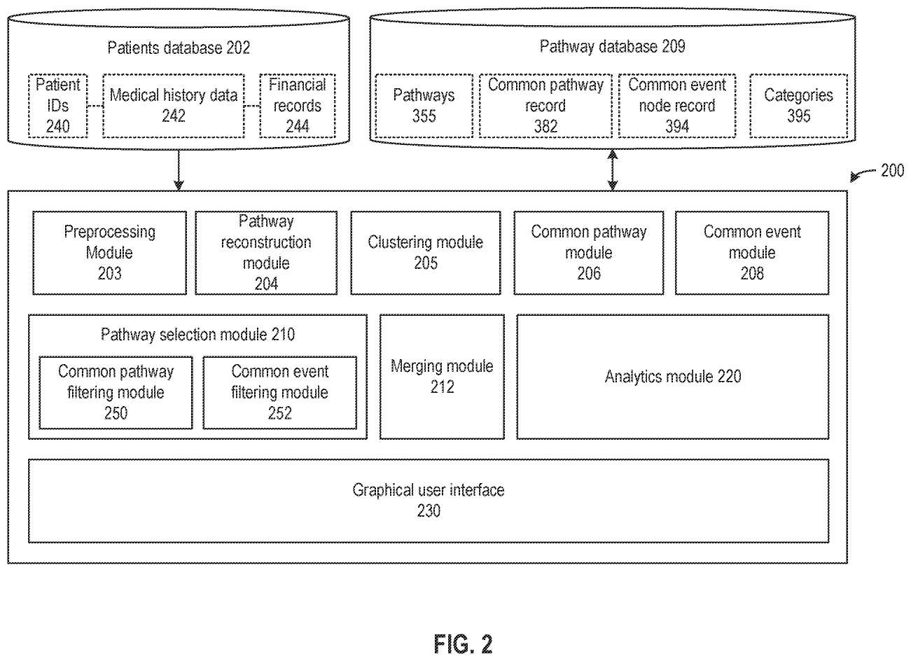

FIG. 2 illustrates an example of a patient pathway reconstruction and aggregation system 200 that can address at least some of the issues described above. Patient pathway reconstruction and aggregation system 200 can receive medical history data of patients from a patients database 202 and reconstruct a patient pathway for each of the patients. Reconstructing an individual pathway can show a patient's care journey over time which helps provide better care to the patient. These pathways can be aggregated on a population level which provides useful information about trends in multiple patients. Various techniques are described for making these aggregated pathways useful and transparent to help provide useful information to clinicians. In some examples, the medical history data may also include an event log. Patient pathway reconstruction and aggregation system 200 can also receive an input to selectively display a subset of the patient pathways.

Patient pathway reconstruction and aggregation system 200 can also compute various metrics for each patient pathway, such as total time and total cost incurred by the patient pathway, total number of patients taking the patient pathway, the survival rate of the patients, a degree of deviation of the patient pathway from a clinical guideline, etc. Patient pathway reconstruction and aggregation system 200 can display the metrics with the selected subset of patient pathways. Patient pathway reconstruction and aggregation system 200 can also support additional analyses, such as identification of different patient cohorts and analysis of characteristics of the patient cohorts, to provide more clinical and operational insights, to support a clinical prediction for a patient whose clinical journey has not yet completed, etc.

Specifically, as shown in FIG. 2, patient pathway reconstruction and aggregation system 200 can include a preprocessing module 203, a pathway reconstruction module 204, a clustering module 205, a common pathway module 206, a common event module 208, a pathway selection module 210, a merging module 212, an analytics module 220, and a graphical user interface 230. Pathway reconstruction module 204 can aggregate various data in patients database 202, such as patient identifiers (ID) 240 that uniquely identify different patients, the patients' medical history data 242, as well as their financial records 244, and generate a graph representing a patient pathway for each patient. Graphical user interface 230 can display some or all of the graphs in a merged graph format, together with various metrics associated with the displayed graphs.

### B. Example Medical History Data

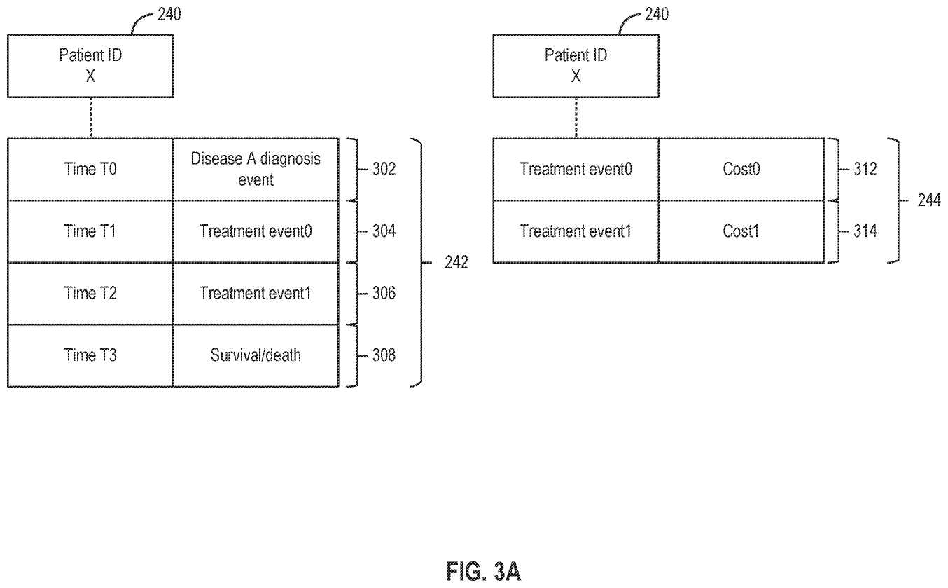

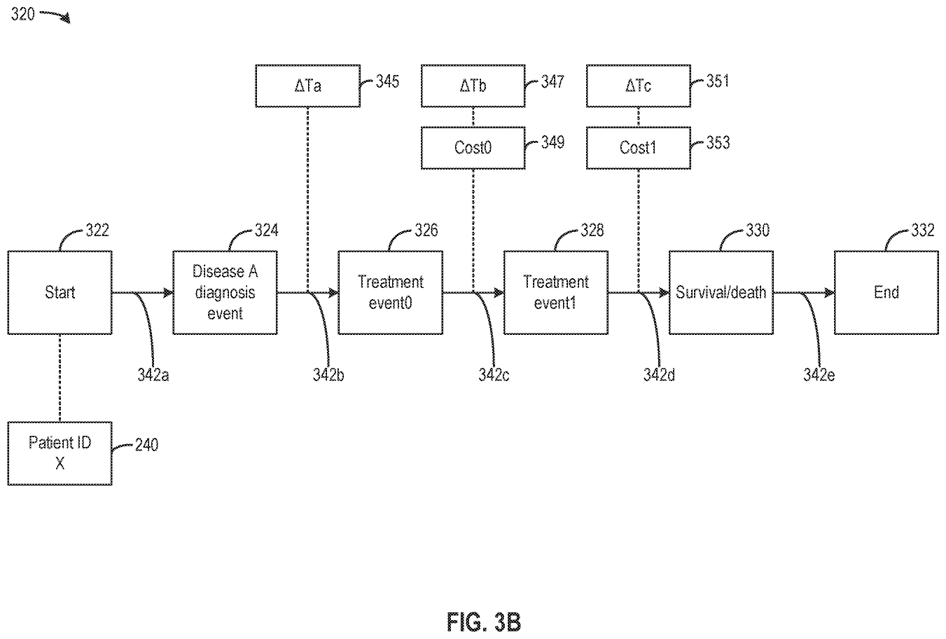

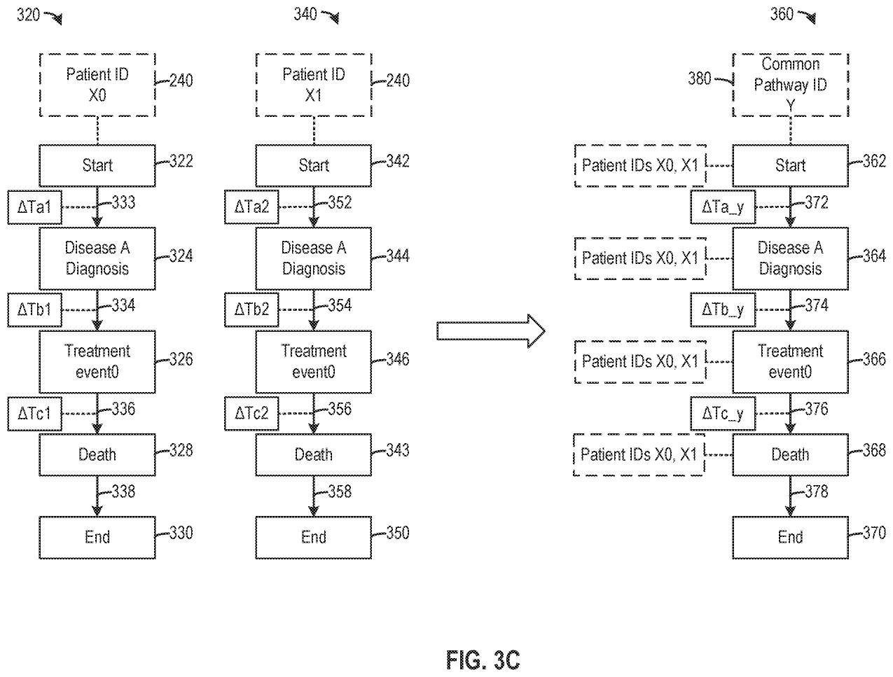

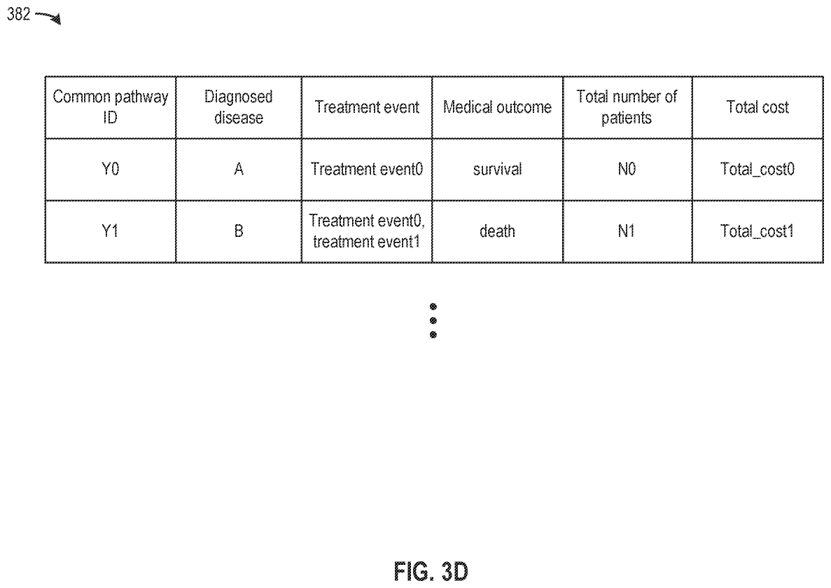

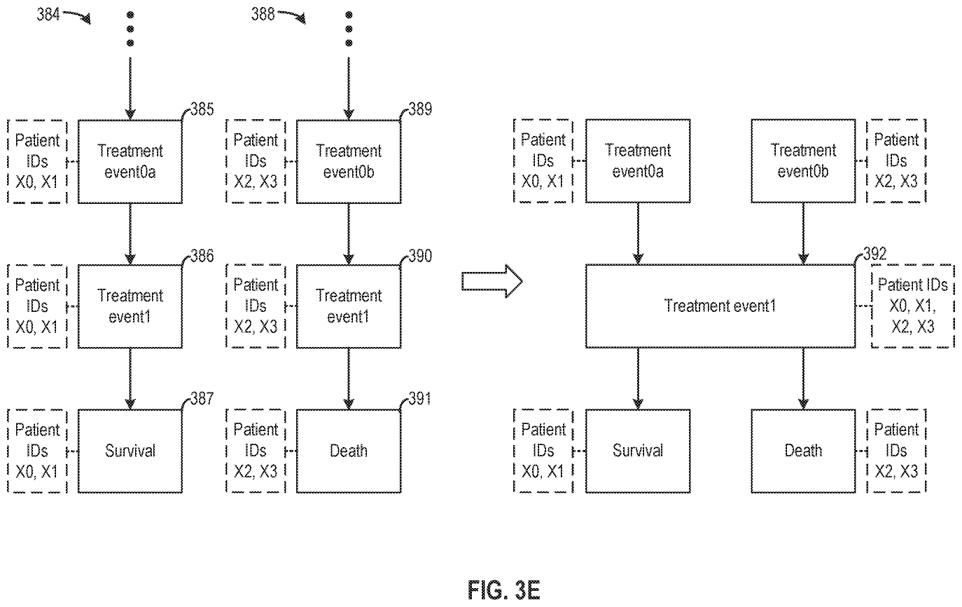

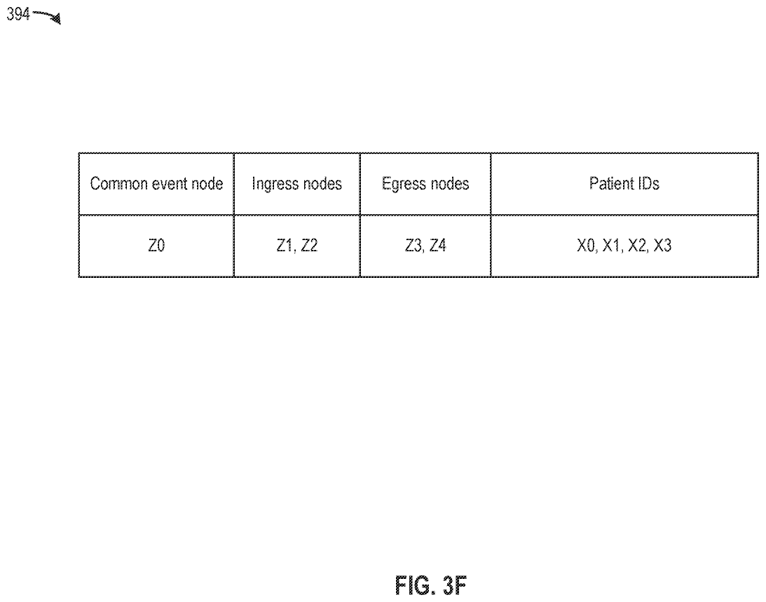

FIG. 3A and FIG. 3B illustrate an example of medical history data 242 and financial records 244, as well as examples of a graph generated by pathway reconstruction module 204. As shown in FIG. 3A, medical history data 242 can be associated with a patient identifier 240 (with a value X) of a patient and can include a data structure (e.g., a mapping table) that stores various events of the patient in the patient's medical history, as well as a medical outcome event (e.g., survival/death), and a time for each event. For example, medical history data 242 may include entries 302 that map a disease A diagnosis event to time T0, entries 304 that map a treatment event labelled event0 to time T1, entries 306 that map a treatment event labelled event1 to time T2, and entries 308 that map a medical outcome event (which can be survival or death) to time T3. Each treatment event may include a therapy regimen, a surgery regiment, etc., each of which can include administering of one or more doses of medications and/or surgery operations over a period of time represented by the mapped time (e.g., times T1, T2, etc.). In addition, financial records 244 can include entries 312 that map the treatment event event0 with a monetary cost represented by cost0, and entries 314 that map the treatment event event1 with a cost represented by cost1.

Preprocessing module 203 can retrieve data from the patients database 202 and prepare the data for further processing. For example, preprocessing module 203 may convert medical history data 242 and/or financial records 244 to tabular format. Preprocessing module 203 may select variables of interest. Preprocessing module 203 can merge events into categories 395. For example, events associated with blood glucose levels, cancer staging, and biopsy results are all grouped into a higher level category 395 for biomarkers. Events associated with administering a particular drug and chemotherapy are categorized into a higher level category 395 for treatment. Within these higher level categories 395 the preprocessing module may also identify lower level categories 395. For example, within biomarkers, there are different types of biomarkers such as radiographic, molecular, histologic, and physiologic. Preprocessing module 203 can identify biomarker events in each category and group the events together for both lower level categories 395 (e.g., molecular biomarkers) and/or higher level categories 395 (e.g., biomarkers).

Pathway reconstruction module 204 can traverse through medical history data 242 and financial records 244 and generate a graph representing a patient pathway undertaken by the patient. The graph can include nodes representing events and directional edges connecting the nodes. Pathway reconstruction module 204 can traverse through medical history data 242 following a temporal order of the events, create a node for each event, and add the nodes and the edges to the graph following the temporal order. Pathway reconstruction module 204 can also compute various metrics, such as time and cost, by traversing through medical history data 242 and financial records 244, and associate the metrics with the edges. Pathway reconstruction module 204 can also store the graphs back to a pathway database 209.

### C. Example Patient Pathway Graph

FIG. 3B illustrates an example of a graph 320 generated by pathway reconstruction module 204 based on medical history data 242 and financial records 244. As shown in FIG. 3B, graph 320 can be associated with patient identifier (ID) 240 (with a value X) and include a start node 322, event nodes including an event node 324 representing a disease A diagnosis event, an event node 326 representing treatment event0, an event node 328 representing treatment event1, an event node 330 representing a medical outcome event (survival or death), and an end node 332. The nodes are also connected with directional edges representing temporal relationships between the nodes and the events. For example, start node 322 is connected to disease A diagnosis event node 324 by an edge 342a. Treatment event0 node 326 is connected to disease A diagnosis event node 326 by an edge 342b, treatment event1 node 328 is connected to treatment event0 node 326 by an edge 342c. medical outcome event node 330 is connected to treatment event1 node 328 by an edge 342d, whereas end node 332 is connected to medical outcome event node 330 by an edge 342e.

Each edge can be an egress edge for a prior node and an ingress edge for a subsequent node, and the direction of the edges also indicate that disease A diagnosis event node 324 precedes treatment event0 node 326, precedes treatment event1 node 328, which in turn precedes medical outcome event node 330. Some of the edges can also be associated with metrics such as time and cost. For example, edge 342b is associated with a transition time 345 elapsed between the disease A diagnosis event and treatment event0, which can be represented by ΔTa (e.g., differences between times T1 and T0 of FIG. 3A). Moreover, edge 342c is associated with a transition time 347 elapsed between treatment event0 and treatment event1, which can be represented by ΔTb (e.g., differences between times T2 and T1 of FIG. 3A), and a cost 349, which can equal to cost0, a monetary cost associated with treatment event0. Further, edge 342d is associated with a transition time 351 elapsed between treatment event1 and the medical outcome event, which can be represented by ΔTc (e.g., differences between times T3 and T2 of FIG. 3A), and a cost 353, which can equal to cost1, a monetary cost associated with treatment event1. Pathway reconstruction module 204 can then store the graphs back to pathway database 209 as pathways 355.

### D. Patient Pathway Clustering

Referring back to FIG. 2, clustering module 205 can cluster patients based on individual patient pathways as generated by the pathway reconstruction module 204. In some implementations, clustering module 205 clusters the patients to build cohorts of patients with similar trajectories. The clusters are built based on similar sequences of events. For example, clustering module clusters the patients based on the order of events in the patient pathway graphs, the time between one or more events in the patient pathway graphs, or other factors that represent the patient trajectory. As another example, the individual patient pathways are compared using Natural Language Processing (NLP) techniques, which can give a similarity score used to identify patients to cluster together. For example, a baseline model, Word Mover's Distance (WMD) combined with Hierarchical Density-Based Spatial Clustering of Applications with Noise (HDBSCAN) or Spectral clustering and Uniform Manifold Approximation and Projection (UMAP) dimensionality reduction can be used to identify similar patient pathways.

### E. Common Pathways

Common pathway module 206 can identify common patient pathways shared by multiple patients, and generate metrics for the common patient pathways. In some implementations, the common pathway module 206 identifies common patient pathways based on the clusters identified by the clustering module 205. Each cluster may be assessed separately, which simplifies the process since the clusters contain patients with similar pathways. As to be described below, a subset of the common pathways can be provided for display by graphical user interface 230. Common pathway module 206 can determine that two patient pathways are identical if two patient pathways have the same sequence of diagnosis event, treatment event, and medical outcome event, with the sequence defined by the ordering of the events in the graphs representing the patient pathways. Each individual patient pathway may include events associated with a particular patient, such as a type of treatment administered to a patient with a particular patient identifier at a particular date, time, and location. These events fall into different event types that are not particular to a particular patient or a specific event. For example, a type of diagnosis event is based on a disease a patient is diagnosed with in an event. As another example, a type of treatment event is based on a therapy administered to a patient or a surgical operation administered to the patient. As another example, a type of clinical outcome event may be based on survival of a patient at a pre-determined time after the one or more treatment events, or death of the patient at the pre-determined time after the one or more treatment events.

### 1. Common Pathway Examples

FIG. 3C illustrates an example operation of common pathway module 206. As shown in FIG. 3C, the patient pathway of a patient with a patient ID 240 (of X0) can be represented by a graph 320 including a start node 322, a disease A diagnosis event node 324, a treatment event0 node 326, a medical outcome (death) node 328, and an end node 330, with the nodes connected by edges 333, 334, 336, and 338. Edge 333 may be associated with a transition time ΔTa1, edge 334 may be associated with a transition time ΔTb1, whereas edge 336 may be associated with a transition time ΔTc1. In addition, the patient pathway of a patient with a patient ID 240 (of X1) can be represented by a graph 340 including a start node 342, a disease A diagnosis event node 344, a treatment event node 346, a medical outcome (death) node 343, and an end node 350, with the nodes connected by edges 352, 354, 356, and 358. Edge 352 may be associated with a transition time ΔTa2, edge 354 may be associated with a transition time ΔTb2, whereas edge 356 may be associated with a transition time ΔTc2.

In the example of FIG. 3C, common pathway module 206 may determine that graphs 320 and 340 represent a common patient pathway based on both graphs having the same sequence of events, comprising an event of a diagnosis of the same disease A (represented by nodes 324 and 344), an event of the same treatment (represented by nodes 326 and 346), and the same medical outcome event (death, represented by nodes 328 and 343). Based on this determination, common pathway module 206 can create a graph 360 representing a common patient pathway based on aggregating the information of graphs 320 and 340. For example, graph 360 includes a common start node 362, a common disease A diagnosis event node 364, a common treatment event0 node 366, a common medical outcome (death) node 368, and a common end node 370, with the nodes connected by edges 372, 374, 376, and 378. Each node is associated with patient ID 240 of patients who share the common patient pathway (e.g., X0, X1). Moreover, each of edges 372, 374, 376, and 378 is formed by merging the edges and the associated information of graphs 320 and 340. For example, edge 372 is associated with a transition time ΔTa_y which can be an average of ΔTa1 and ΔTa2. Moreover, edge 374 is associated with a transition time ΔTb_y which can be an average of ΔTb1 and ΔTb2. Further, edge 376 is associated with a transition time ΔTc_y which can be an average of ΔTc1 and ΔTc2. Other information associated with the edges, such as cost (not shown in FIG. 3C), can also be averaged and become associated with the merged edges in graph 360. Graph 360 is also associated with a common path ID 380 (labelled Y in FIG. 3C).

### 2. Common Pathway Record Examples

After identifying the common pathways, common pathway module 206 can generate a record (e.g., in the form of a table) that associates various metrics and events with each common pathway, and store the record in pathway database 209. FIG. 3D illustrates an example of a common pathway record 382. As shown in FIG. 3D, common pathway record 382 may associate each common pathway ID (e.g., Y0, Y1, etc.) with the diagnosis events, the treatment events, the medical outcome events, a total number of patients who share the common patient pathway associated with the ID (e.g., N0, N1, etc.) and a total cost of the common patient pathway (e.g., total_cost0, total_cost1, etc.). The total number of patients for each common patient pathway can be obtained by counting the number of unique patient identifiers associated with a common event node of the graph (e.g., graph 360) representing the common patient pathway, whereas the total cost for each common patient pathway can be obtained by summing the average cost associated with each merged edge of the graph. Common pathway record 382 can also be stored in pathway database 209. In some examples, common pathway record 382 can also be created by counting edges that transit between nodes representing common diagnosis events, the treatment events, the medical outcome events.

### F. Common Events

Referring back to FIG. 2, common event module 208 can identify common event nodes (diagnosis, treatment, medical outcome) that are shared by patients who do not share a common patient pathway, and create a record that associate a common event node with patient IDs of the patients having the common event node as part of their patient pathways, as well as other event nodes connected to the common event node.

FIG. 3E and FIG. 3F illustrates an example operations of common event module 208. Common event module 208 can traverse through the graphs of patient pathways stored in pathways 355 and/or common pathway record 382. FIG. 3E illustrates a graph 384 associated with patient IDs X0 and X1 and comprising node 385 associated with treatment event0a, node 386 associated with treatment event1, node 387 associated with a medical outcome event (survival), a graph 388 associated with patient IDs X2 and X3 and comprising node 389 associated with treatment event0b, node 390 associated with treatment event1, and node 391 associated with a medical outcome event (death).

Common event module 208 can determine that both graphs have a node representing treatment event1 (nodes 386 and 390), and create a common event node 392 representing treatment event1 with ingress edges from treatment event nodes 385 and 389 and egress edges to medical outcome event nodes 387 and 391. Common event module 208 can also associate common event node 392 with patient IDs X0, X1, X2, and X3. In addition, referring to FIG. 3F, common event module 208 can also create a common event node record 394 (e.g., a table) that associate each common event node with a common event node ID, ingress nodes (from which the ingress edges are connected) and egress nodes (to which the egress edges are connected), as well as the patient IDs associated with the common event node. In a case where the common event node is associated with a treatment, common event node record 394 may further store information about a specific treatment regime and/or a specific surgery regime associated with the common event node. For example, in FIG. 3F, in common event node record 394 common event node 392 is assigned a common event node ID “Z0” and is associated with patient IDs X0, X1, X2, and X3. Common event node 392 is further associated with treatment event nodes 385 and 389 (represented by event node IDs “Z1” and “Z2”) as ingress nodes, and with medical outcome event nodes 387 and 391 (represented by event node IDs “Z3” and “Z4”) as egress nodes. Common event node record 394 can be stored in pathway database 209 as well.

### G. Pathway Selection

Referring back to FIG. 2, pathway selection module 210 can receive an input that specifies one or more selection criteria, and select a subset of the patient pathways based on the selection criteria, retrieve the graphs representing the selected patient pathways from pathway database 309, and then provide the graphs for displaying in graphical user interface 230. In some examples, the input can be received via graphical user interface 230 as well.

Pathway selection module 210 can include a common pathway filtering module 250 and a common event filtering module 252 to perform the selection based on different types of criteria. Specifically, common pathway filtering module 250 can receive an input that specifies a number (e.g., four, eight, ten, etc.) of the most common patient pathways shared by the patients. Common pathway filtering module 250 can then refer to common pathway record 382 and identify the number of common patient pathways associated with the most number of patients, rank the common patient pathways based on the number of patients, and retrieve the graphs representing the number of top-ranked common patient pathways. As another example, the input may specify that only a percentage of patients (e.g., 100%, 70%, 50%, 10%, etc.) are to be represented in the common patient pathways. Common pathway filtering module 250 can also instruct common pathway module 206 to generate samples of common patient pathways based on samples of patient pathways of the specified percentage of patients, and provide the graphs of the samples of common patient pathways for displaying in graphical user interface 230. In some examples, pathway selection module 210 can also obtain the metrics data of the common patient pathways from common pathway record 382 and provide them for displaying in graphical user interface 230.

In addition, common event filtering module 252 can receive an input that specifies criteria for selecting one or more common treatment events, and the system can select the subset of patient pathways having the one or more common treatment events. For example, the criteria may specify a specific treatment regime or a specific surgery regime. Common event filtering module 252 can refer to common event node record 394 and identify a common treatment event node having the specified treatment/surgery regime, and identify the ingress and egress nodes of the identified common treatment event node. Common event filtering module 252 can also trace the ingress and egress nodes of the identified nodes in common event node record 394 until reaching the nodes representing the diagnosis events and the medical outcome events. As another example, the criteria may specify that a common treatment event shared by a threshold percentage of patients, or a threshold percentage of patients transition from a first common treatment event to a second common treatment event, and common event filtering module 252 can filter out transitions/edges that do not satisfy the threshold percentage as part of the tracing. Common event filtering module 252 can then provide graphs representing the nodes identified from common event node record 394, including the treatment event nodes, the diagnosis event nodes, and the medical outcome event nodes, to graphical user interface 230 for displaying.

In some examples, merging module 212 can receive the graphs provided by pathway selection module 210 and merge them into a merged graph for displaying in graphical user interface 230. The merging operation can include merging nodes of different graphs representing a same type of diagnosis event, a same type of treatment event, or a same type of clinical outcome event into a merged nodes, and based on merging the edges between two merged nodes into a single edge. By merging the graphs, the merged graph can become more compact and can be more easily visualized.

### H. Merging

In some examples, merging module 212 merges the graphs based on categories determined by preprocessing module 203. Merging module 212 can create a pathway based on higher level categories (e.g., biomarkers). Merging module 212 can nest lower level categories (e.g., molecular biomarkers) within the pathways, and populate individual biomarkers therein.

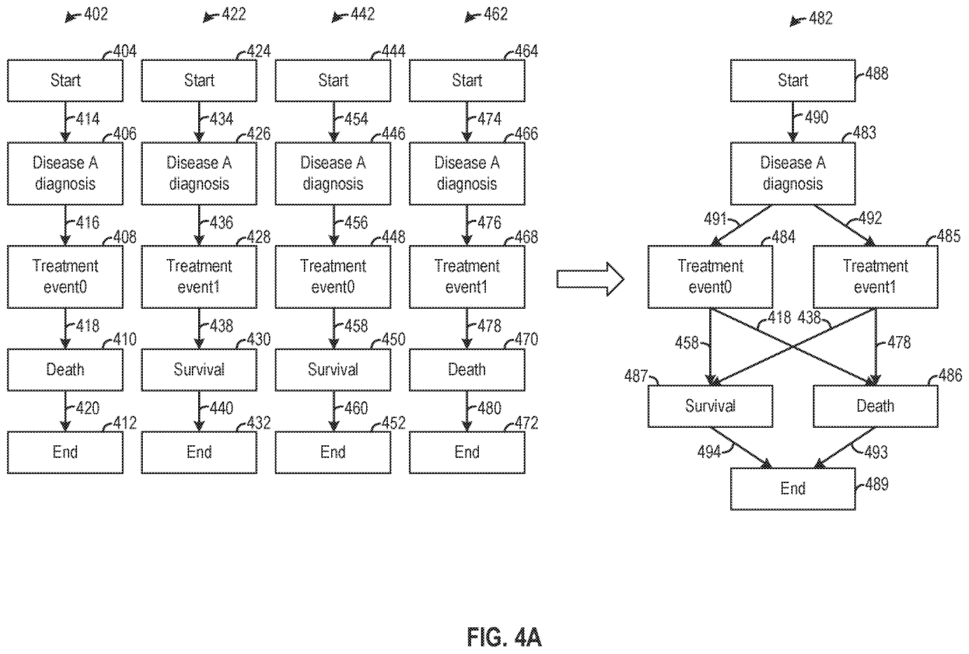

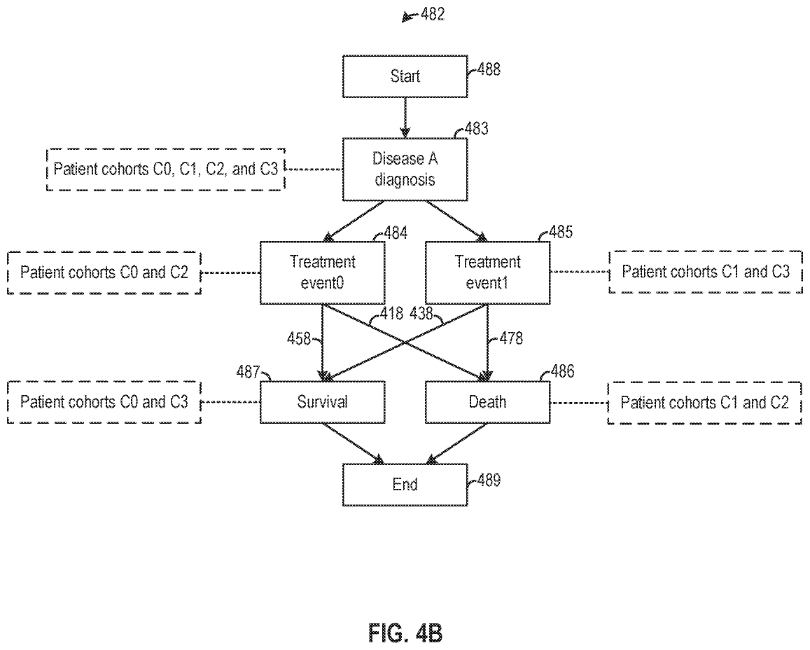

FIG. 4A illustrates an example merging operation by merging module 212. As shown in FIG. 4A, pathway selection module 210 can provide a graph 402, a graph 422, a graph 442, and a graph 462. Each graph can represent the patient pathway of a single patient, or a common patient pathway generated by common pathway module 206. Graph 402 includes a start node 404, a disease A diagnosis event node 406, a treatment event0 node 408, a medical outcome event (death) node 410, and an end node 412, connected by edges 414, 416, 418, and 420. Graph 422 includes a start node 424, a disease A diagnosis event node 426, a treatment event1 node 428, a medical outcome (survival) node 430, and an end node 432, connected by edges 434, 436, 438, and 440. Further, graph 442 includes a start node 444, a disease A diagnosis event node 446, a treatment event0 node 448, a medical outcome event (survival) node 450, and an end node 452, connected by edges 454, 456, 458, and 460. In addition, graph 462 includes a start node 464, a disease A diagnosis event node 466, a treatment event1 node 468, a medical outcome event (survival) node 470, and an end node 472, connected by edges 474, 476, 478, and 480.

Merging module 212 can traverse through graphs 402, 422, 442, and 462, and merge them into a merged graph 482. The merging operation can include merging nodes representing a same diagnosis event, a same treatment event, or a same clinical outcome event into a merged node, and based on merging the edges between two merged nodes into a single edge. As shown in FIG. 4A, merging module 212 can merge nodes 406, 426, 446, and 466, each representing the same disease A diagnosis event, into a merged node 483 representing the disease A diagnosis event. In addition, merging module 212 can merge nodes 408 and 448 representing treatment event0 into a merged node 484, and merge nodes 428 and 468 representing treatment event1 into a merged node 485. Further, merging module 212 can merge nodes 410 and 470 representing a death event into a merged node 486, and merge nodes 430 and 450 representing a survival event into a merged node 487. Merging module 212 can also merge the start nodes and the end nodes into, respectively, a single start node 488 and a single end node 489. In some implementations, additional information is stored in a node, such as a number of patients associated with that node.

In addition, merging module 212 also merges the edges. As part of the merging, metrics associated with the edges, such as time, cost, total number of patients, etc., can be combined (e.g., averaged, added, etc.) into the merged edges. For example, edges 414, 434, 454, and 474 are merged into an edge 490, edges 416 and 456 can be merged into an edge 491, edges 436 and 476 can be merged into an edge 492, edges 420 and 480 are merged into an edge 493, whereas edges 440 and 460 are merged into an edge 494. On the other hand, edges 418, 438, 458, and 478 between treatment event nodes and medical outcome event nodes are retained in merged graph 482.

In some examples, merging module 212 can also associate each event node with the patient IDs of the patients who share the event node. As to be described below, the association of patients with the shared event nodes can support additional analyses and visualization effects. For example, referring to FIG. 4B, assuming that cohorts of patients C0, C1, C2, and C3 shares, respectively, patient pathways represented by graphs 402, 422, 442, and 462, merging module 212 can associate disease A diagnosis event node 483 of merged graph 482 with cohorts C0, C1, C2, and C3. Moreover, merging module 212 can associate treatment event0 node 484 with cohorts C0 and C2, and associate treatment event1 node 485 with cohorts C1 and C3. Merging module 212 can also associate survival event node 487 with cohorts C0 and C3, and death event node 486 with cohorts C1 and C2.

### I. Analytics

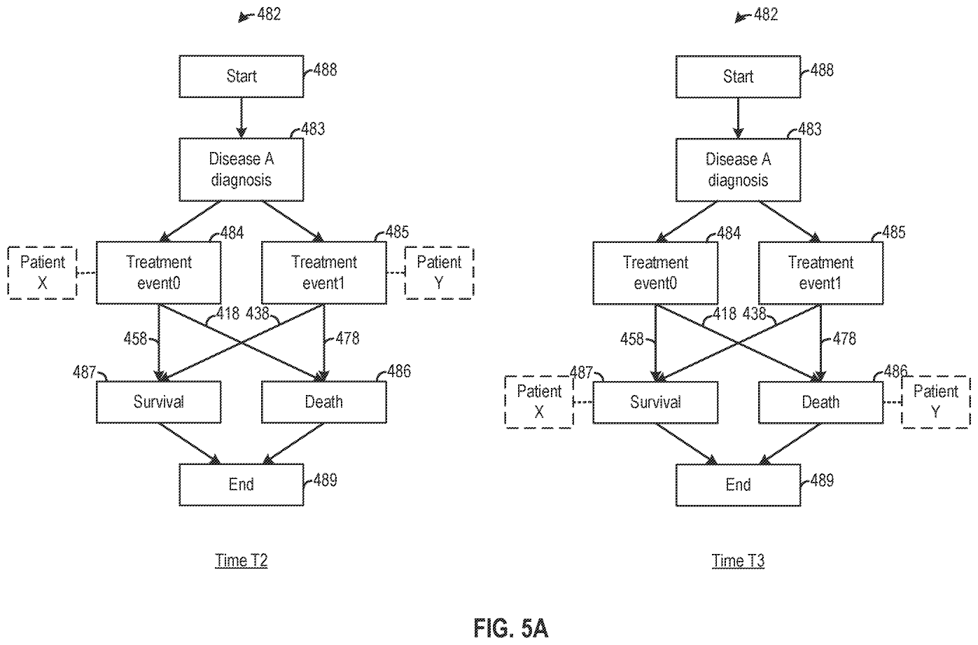

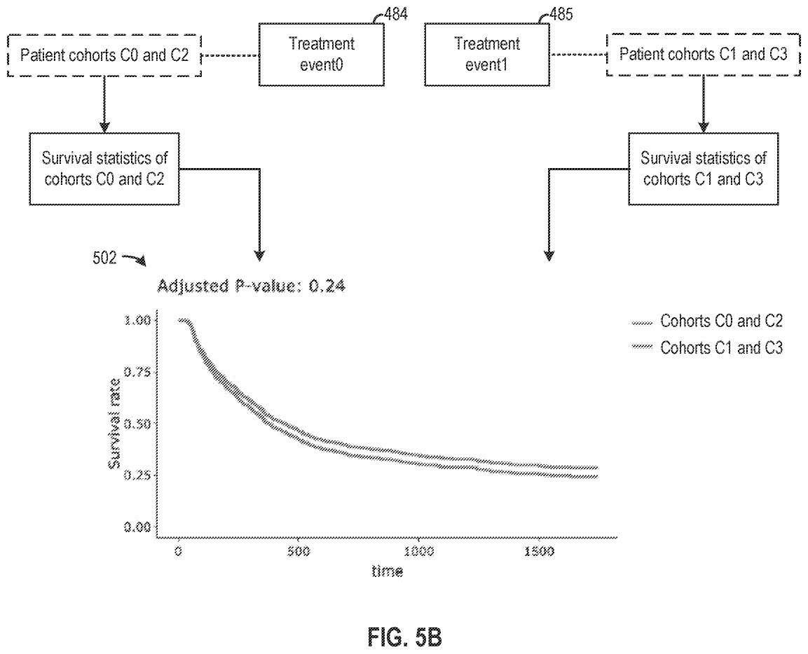

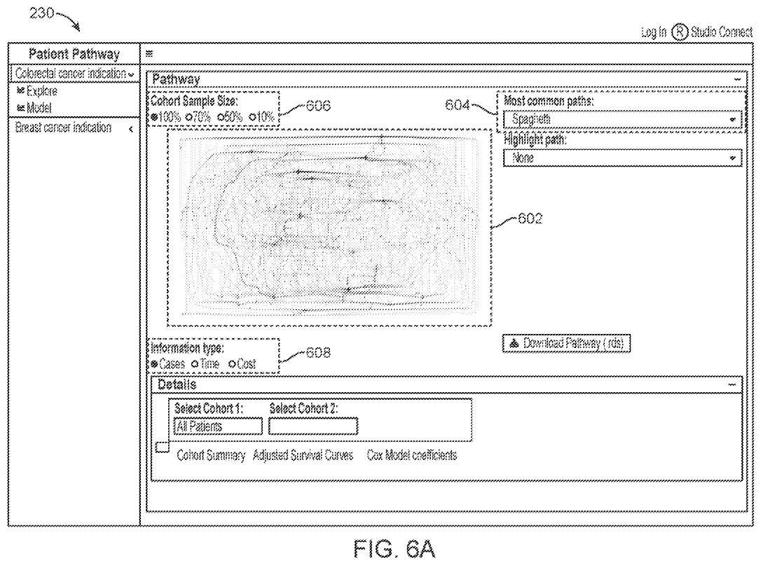

Analytics module 220 can perform additional analyses on the merged graph generated by merging module 212. For example, referring to FIG. 5A, analytics module 220 can track the progress of each patient (or each patient cohort) represented in merged graph 482 with time. The tracking can be based on the association of the patients with each node in the merged graph, as well as the time information included in the edges. In some examples, graphical user interface 230 can be configured to display the progress of the patients in merged graph 482 with respect to time. For example, graphical user interface 230 can display that patients X and Y are at, respectively, treatment event0 node 484 and treatment event1 node 485 at time T2. Moreover, graphical user interface 230 can also display that patients X and Y are at, respectively, survival event node 487 and death event node 486 at time T3. The output of analytics module 220 in FIG. 5A can support an animated display of merged graph 482 to show the progress of the patients in the graph with respect to time, to improve the visualization of various metrics (e.g., time, survival rate, etc.) of different patient pathways.

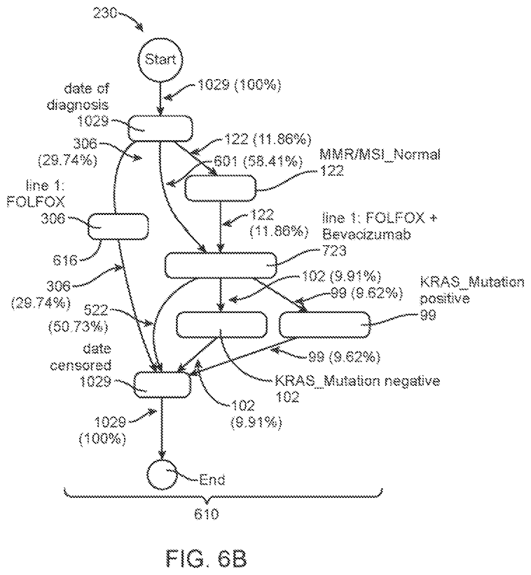

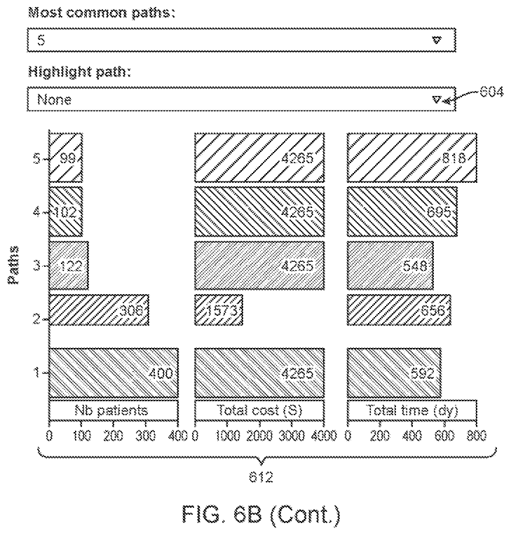

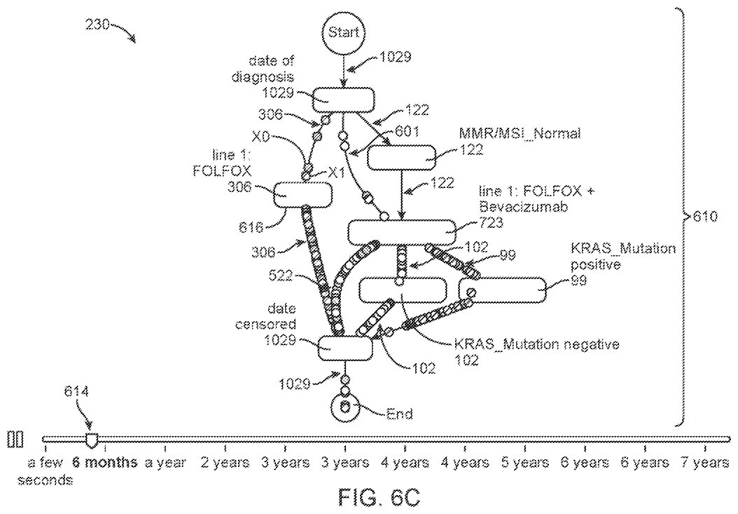

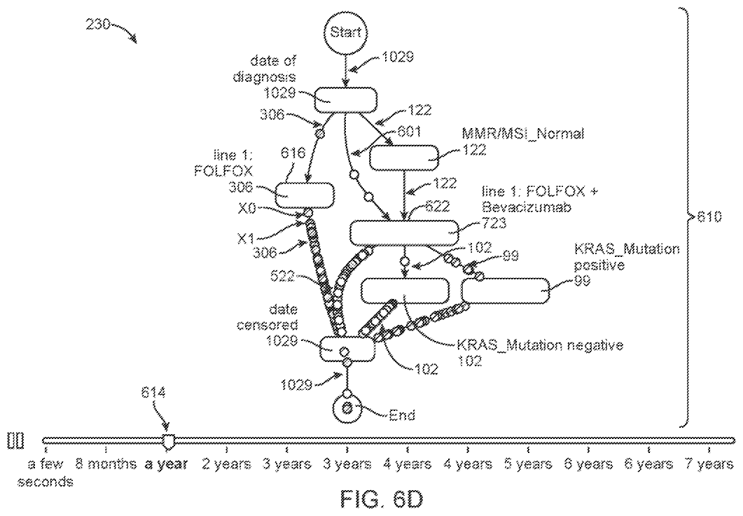

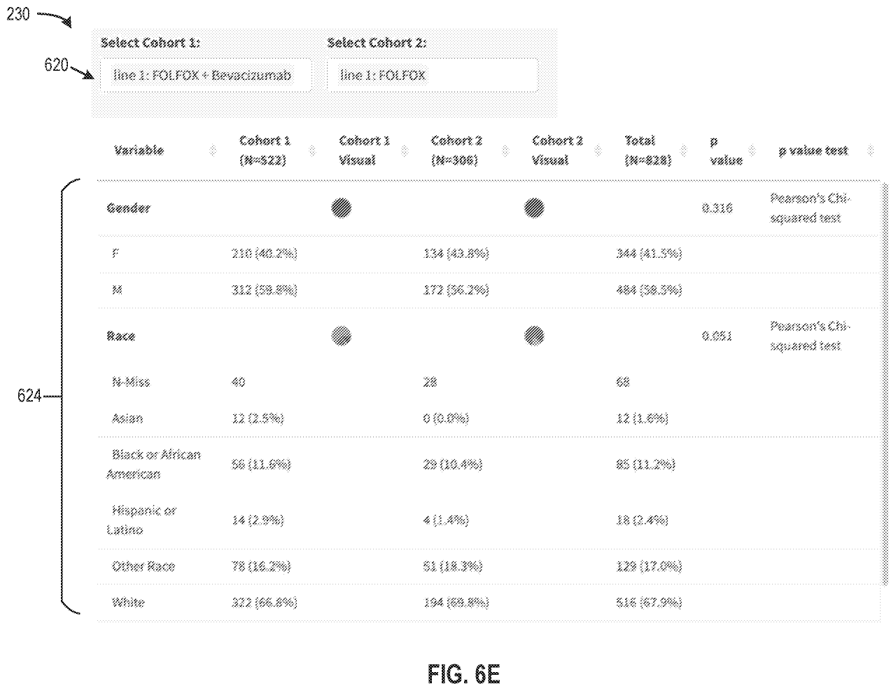

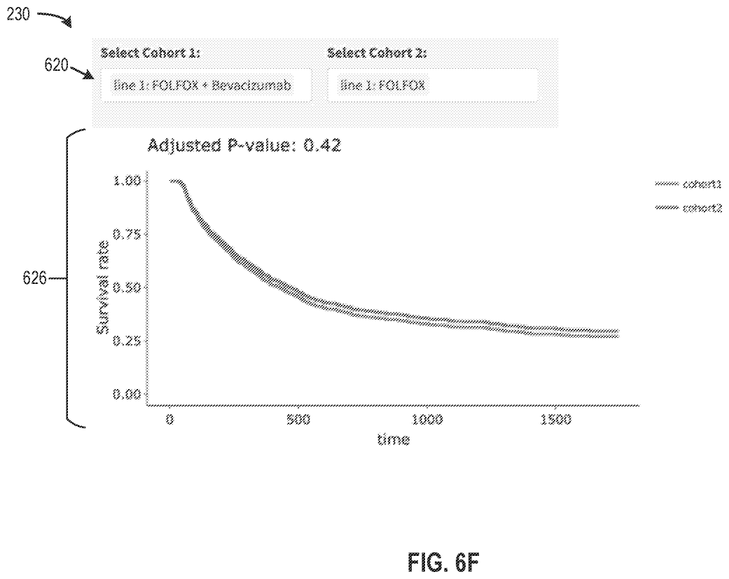

In some examples, analytics module 220 uses the categories 395 generated by the preprocessing module 203 to generate nested pathway visualizations. Using the higher level and/or lower level categories generated by preprocessing module 203, analytics module 220 can organize the pathways to be displayed based on categories. This can be used to produce a more user-friendly visualization that clearly shows the different kinds of events in the pathway (e.g., as shown in FIG. 6G). In some implementations, a user (e.g., a doctor) can scroll or zoom into a higher level category, such as biomarker testing, and view the different types of biomarkers in the pathway.

In some examples, analytics module 220 can access medical history data of patients associated with a particular event node to support additional analyses, such as survival rate analysis. For example, referring to FIG. 5B, treatment event0 node 484 and treatment event1 node 485 can be discriminating nodes associated with mutually exclusive cohorts of patients. Analytics module 220 can retrieve the medical history data of cohorts C0 and C2 of patients associated with treatment event0 node 484, and the medical history data of cohorts C1 and C3 of patients associated with treatment event1 node 485, and generate Kaplan-Meier (K-M) plots 502. In some examples, adjusted Cox regression model curves can also be generated. The K-M plots of the cohorts involved in the treatments can then be analyzed to determine, for example, the effectiveness of the treatment/surgery regimes represented by nodes 484 and 485. Further, analytics module 220 may also perform a prediction based on K-M plots 502. For example, for a patient who is deciding between treatment options represented by nodes 484 and 485, the characteristics of the patient can be compared with those of cohorts C0, C1, C2, and C3, and the K-M plot for the cohort having the most similar characteristics as the patient can be used to, for example, generate hypothesis to enable a prediction of the patient's survival rate after taking up the same treatment taken by the cohort. These interactive features can be used to assist a clinician make informed decisions to investigate the relevant attributes and to determine courses of actions (e.g., treatments) to improve the probability of survival of the patient and/or improve the patient's ability to plan for the future and improve the patient's quality of life. By interacting with the interface to view additional analytics, the clinician can identify potential deviations in clinical care from clinical guidelines and potential inefficiencies in clinical workflow and resource management by navigating the interface to view the additional metrics.

### III. Graphical User Interface

FIG. 6A-FIG. 6F illustrate examples of operations of graphical user interface 230. As shown in FIG. 6A, graphical user interface 230 can include a display interface 602 to display a merged graph of patient pathways, as well as input interfaces 604, 606, and 608. Input interface 604 can provide input about a number of most common patient pathways to be displayed in display interface 602, whereas input interface 606 can provide input about a percentage of patients to be represented in the common patient pathways. Moreover, input interface 608 can provide input to select the metrics (e.g., number of patients (cases), time, and cost) to be displayed in the merged graph. In FIG. 6A, graphical user interface 230 is configured to display a fully merged graph of all the patient pathways of all patients in patients database 202.

FIG. 6B illustrates another example operation of graphical user interface 230. As shown in FIG. 6B, graphical user interface 230 can receive an input from input interface 604 to display the top five most common patient pathways as a merged graph 610, based on the outputs of common pathway module 206, pathway selection module 210, and merging module 212, and an input from interface 608 to display the number of patients associated with each node and edge. Graphical user interface 230 also displays a graph 612 of various metrics, including total number of patients, total cost, and total time, for each common patient pathway.

FIG. 6C and FIG. 6D illustrate example operations of graphical user interface 230 based on the outputs of analytics module 220. As shown in FIG. 6C and FIG. 6D, graphical user interface 230 can operate in an animated mode based on outputs of analytics module 220. Graphical user interface 230 further includes an input interface 614, which can be in the form of a time slider, to select a time of the progress of the patients in the common patient pathway represented by merged graph 610. In FIG. 6C and FIG. 6D, each dot on the edges (e.g., X0 and X1) can represent a case/patient, and the patient moves from one event node to another event node in merged graph 610 between different times selected by input interface 614. For example, as shown in FIG. 6C, patients X0 and X1 have not yet started a FOLFOX treatment (a chemotherapy regimen made up of the drugs Folinic acid (leucovorin), Fluorouracil (5-FU), and Oxaliplatin (Eloxatin)) represented by treatment event node 616 at 6 months from the diagnosis of the disease, whereas in FIG. 6D, patients X0 and X1 have completed the FOLFOX treatment at 1 year from the diagnosis of the disease.

FIG. 6E and FIG. 6F illustrate other example operations of graphical user interface 230 based on the outputs of analytics module 220. As shown in FIG. 6E, graphical user interface 230 includes an input interface 620 which receives an input to select two discriminating nodes to identify two patient cohorts. The two discriminating nodes can be two treatment event nodes, two diagnostics nodes, etc. Referring back to FIG. 6D, treatment event node 616 is associated with a FOLFOX treatment, whereas treatment event node 622 is associated with a FOLFOX and Bevacizumab treatment, and each involve a mutually exclusive set of patients. Based on the selection of treatment event nodes 616 and 622, two cohorts (Cohort 1 and Cohort 2) are identified, and their medical data can be accessed by analytics module 220. In FIG. 6E, demographic information 624 of the two cohorts are displayed, whereas in FIG. 6F, K-M plots 626 of the two cohorts are displayed.

FIG. 6G illustrates another example of a view of graphical user interface 230 showing nested pathways. In order to improve the visual presentation of pathway maps (e.g., as shown in FIG. 6B), patient pathway reconstruction and aggregation system 200 groups nodes belonging to different categories. For example, as depicted in FIG. 6G, nodes are grouped into three high level categories—line of treatment A 632, biomarkers 634, and line of treatment 2 636. Within each of these high level categories 632, 634, 636, there are respective events. Events A and E are specific first lines of treatment (e.g., different drugs or therapies administered), events B, C, H, I, and F are specific biomarkers (e.g., blood glucose, prostate specific antigen, etc.), and events D and G are specific second lines of treatment.

In the example depicted in FIG. 6G, the node connections can be generated as described above, based on an ordered series of events in multiple patient pathways. Additionally, the nodes are grouped to reflect the different levels of categories. By showing the events grouped in different categories, a more user-friendly visualization is provided that clearly shows the different kinds of events in the pathway. A user (e.g., a doctor) can scroll or zoom into a higher level category, such as biomarker testing, and view the different types of biomarkers in the pathway. This helps the user to drill down into different levels and easily navigate the graph to obtain information of interest.

### IV. Method

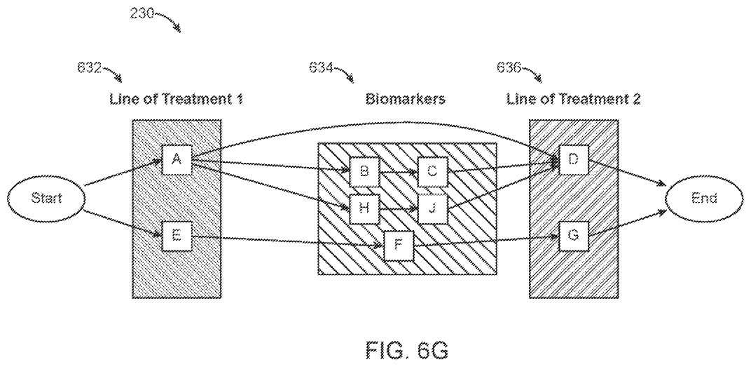

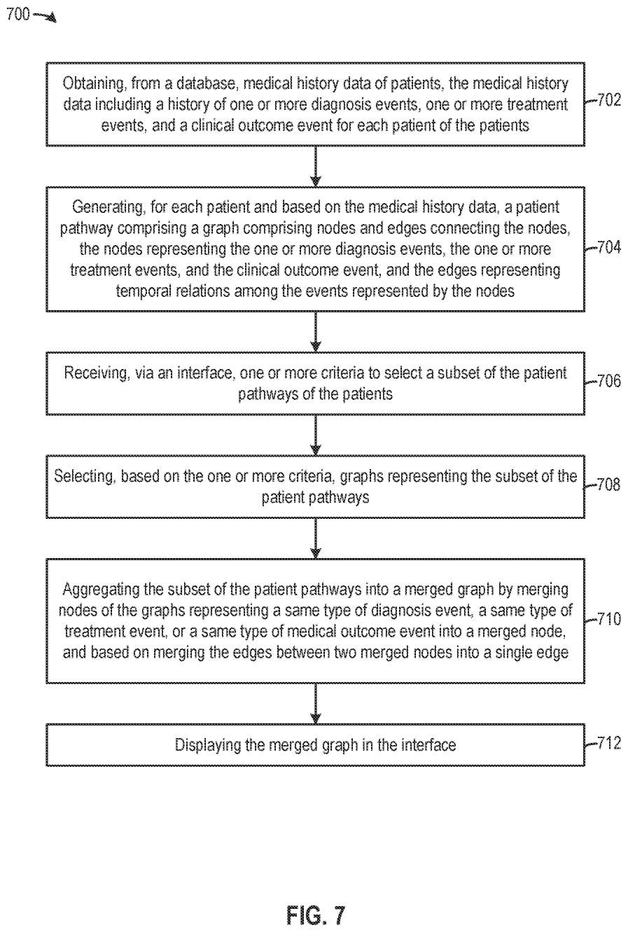

FIG. 7 illustrates an example of a method 700 of reconstructing and aggregating patient pathways of a plurality of patients, based on medical history data of the patients. Method 700 can be performed by, for example, patient pathway reconstruction and aggregation system 200 of FIG. 2.

In step 702, the system obtains, from a database (e.g., patients database 202), medical history data of patients, the medical history data including a history of one or more diagnosis events, one or more treatment events, and a clinical outcome event for each patient of the patients. A diagnosis event can include, for example, an event in which a patient is diagnosed with a disease. A treatment event can include, for example, a therapy regimen, a surgery regiment, etc., each of which can include administering of one or more doses of medications and/or surgery operations over a period of time. The clinical outcome event can include, for example, survival or death at a pre-determined time from the diagnosis, which can be based on a specific study period and adjusted for year of diagnosis. The medical history data can include a large amount of complex data. For example, the medical history data can include data for more than 100 patients, more than 500 patients, more than 1,000 patients, or more than 5,000 patients. For each patient, the medical history data can include dozens or even hundreds of different events.

In some implementations, the system preprocesses the medical history data, which may include formatting the data (e.g., in tabular form, standardizing certain numbers or terms, etc.). In some examples, the preprocessing includes grouping the events into categories. This can include higher level categories such as treatments, biomarkers, etc. Alternatively, or additionally, this can include lower level categories such as molecular biomarkers, drug treatments, etc.

In step 704, the system generates, for each patient and based on the medical history data, a patient pathway comprising a graph comprising nodes and edges connecting the nodes, the nodes representing the one or more diagnosis events, the one or more treatment events, and the clinical outcome event, and the edges representing temporal relations among the events represented by the nodes. FIG. 3B illustrates an example of a patient pathway generated by the system. In some examples, the patient pathway reconstruction and aggregation system can traverse through a medical history record of a patient that arranges the events following a temporal order, create a node for each event, and add the nodes to the graph following the temporal order. As noted above, there can be thousands of events, resulting in thousands of nodes connected by thousands of edges.

In some implementations, the system clusters the patients based on a sequence of events in the patient pathway for each patient to produce patient clusters. For example, trace clustering is applied to the sequence of events in the patient pathways to create clusters of patients following similar sequences. Part of the reason for the spaghetti-like appearance of traditional multi-patient pathways (e.g., as shown in FIG. 6A) is the variability of the patient data, which arises from both the size of the dataset and patients having different treatment paths. By clustering “similar” patients together before applying the pathway building algorithm, the system creates smaller groups of similar patient treatment paths.

In step 706, the system receives, via an interface, one or more criteria to select a subset of the patient pathways of the patients. A user interacts with the interface to configure the criteria. FIG. 6B illustrates an example of the interface. For example, the criteria may specify that all graphs, or all patients, are included in the merged graph. The criteria may specify that only a percentage of patients (e.g., 100%, 70%, 50%, 10%, etc.) are to be represented in the merged graph. As another example, the criteria may also specify a number (e.g., four, eight, ten, etc.) of the most common patient pathways shared by the patients. In some examples, the input may include criteria for selecting one or more common treatment events. For example, the criteria may specify a specific treatment regime or a specific surgery regime. As another example, the criteria may specify that a common treatment event is shared by a threshold percentage of patients, or that a threshold percentage of patients transition from a first common treatment event to a second common treatment event.

In step 708, the system selects, based on the one or more criteria, graphs representing the subset of the patient pathways. Based on the specified criteria, the system identifies and selects corresponding graphs. For example, the criteria specifies a percentage of patients to be represented in the merged graph, and the system selects the subset of the graphs representing the specified percentage of patients. As another example, the criteria specifies one or more common treatment events. The system can select the graphs representing the subset of patient pathways having the one or more common treatment events. As another example, the criteria specifies a specific treatment regime or a specific surgery regime, and the system selects a graph having a treatment event node of the specific treatment regime or the specific surgery regime.

In some examples, the one or more criteria specify a number of patients. The system identifies one or more common patient pathways shared by that number of patients. The system associates each common patient pathway with a count of the number of patients sharing the common patient pathway. The system ranks each patient pathway based on the associated count. The system selects a number of top-ranked patient pathways as the subset of the patient pathways.

In some examples, the one or more criteria specify a threshold percentage of patients. The system identifies one or more common types of treatment event shared by a number of patients. The system associates each common type of treatment event with a percentage of the patients sharing the common type of treatment event. The system selects the subset of the patient pathways including one or more common types of treatment event associated with the specified threshold percentage of the patients.

In some examples, the system selects events for inclusion in the subset of the patient pathways based on identifying common types of events. For example, the system identifies one or more common types of treatment events, which are shared by a number of the patients. The system associates each common type of treatment event with a subset of the patients sharing the common type of treatment event. The system selects a first common type of treatment event and a second common type of treatment event to be included in the subset of the patient pathways. The first common type of treatment event is shared by a first subset of the patients, and the second common type of treatment event is shared by a second subset of the patients who also have the first common type of treatment event prior to having the second common type of treatment event. The first common type of treatment event and the second common type of treatment event are selected to be included in the subset of the patient pathways based on a percentage between the second subset and the first subset exceeding a threshold percentage.

In some implementations, the graphs representing the subset of the patient pathways are further selected based on the patient clusters. For example, one patient cluster is selected, and step 710 is performed for that patient cluster, then another patient cluster is selected and step 710 is performed for that patient cluster, and so forth. This can simplify and improve the merging process since the clusters of patients have similar patient pathways.

In step 710, the system aggregates the subset of the patient pathways into a merged graph by merging nodes of the graphs representing a same type of diagnosis event, a same type of treatment event, or a same type of clinical outcome event into a merged node, and based on merging the edges between two merged nodes into a single edge. Examples of the aggregation are shown in FIG. 3C.

The system identifies, among the subset of the patient pathways, a plurality of sets of nodes of the graphs, each set of nodes representing a same type of diagnosis event, a same type of treatment event, or a same type of clinical outcome event, and merges each set of the plurality of sets of nodes of the graphs. Specifically, the system can determine that two nodes of two diagnosis events of two patients represent the same type of diagnosis event if the two patients have the same diagnosis (e.g., the same type of cancer and at the same stage), and merge the two nodes into a merged node representing the same diagnosis event. Moreover, the system can determine that two nodes of two treatment events of two patients can be determined as representing the same type of treatment event if the two events have the same therapy/surgery regimes, and merge the two nodes into a merged node representing the same treatment event. Further, the system can also merge two nodes of identical types of clinical outcome events (e.g., survival or death), and merge the two nodes into merged node representing the same clinical outcome event. In all these cases, two nodes can be merged whether or not the events represented by the nodes occur within the same or different time periods. For example, nodes representing the same type of diagnosis event, the same type of treatment event, or the same type of clinical outcome event represent events occurring at different times. In some examples, the merging can also include clustering a pair of nodes representing different diagnosis/treatment events that are close in time and/or connected by a large number of edges, or nodes that are selected as part of the input via the graphical user interface, into a single node.

In some implementations, the system generates the merged graph based on categories of events. For example, the system generates a pathway of higher level categories as shown in FIG. 6G, and within these higher level categories, biomarker events are grouped together, treatment events are grouped together, and so forth.

In step 712, the system displays the merged graph in the interface. Examples of the merged graph are shown in FIG. 6A-FIG. 6D. In addition to the merged graph, the system can also generate and display additional analytics data of the subset of patient pathways, which can provide clinical insights and operational insights into the clinical care received by the patients. For example, the system can generate various metrics for each patient pathway and/or for each event node represented in the merged graph, and display the metrics in the interface. The metrics can include, for example, a number of patients who share the patient pathway, a total cost of one or more diagnosis events and one or more treatment events of the patient pathway, a total time between a first diagnosis event and a clinical outcome event in the patient pathway, survival rate of the patients, and deviation (if any) from a clinical guideline. The metrics can be overlaid on the merged graph and/or displayed in a separate graph.

In some examples, the system displays the merged graph with nested pathways (e.g., as shown in FIG. 6G. The merged graph includes higher level categories, event level nodes, and in some cases lower level categories. The system can accept user input to zoom out to higher level categories or zoom in into event level nodes. This improves the visual presentation and interpretability of pathway maps.

In some examples, the system can also support the identification and analysis of different patient cohorts. For example, each node of a common event in the merged graph can be associated with a cohort of patients (e.g., a group of similar patients identified from the merged graph). The medical history data of the subset of the patients can be accessed from the database via a selection of the node at the interface. Various analyses can be performed on the data for the cohort of patients.

In some examples, the system can perform a clinical prediction based on a processing result. For example, the system receives medical data of a patient, compares the medical data of the patient with medical data of the first subset of patients, and performs a clinical prediction for the patient based on the first processing result and a result of the comparison. For example, an identified cohort of patients is used to predict a probability of survival. As a specific example, a predictive machine learning model can be trained to perform a clinical prediction to predict a medical outcome for a particular patient. For example, a random survival forest (RSF) model can be trained based on the data of previous patients, as well as their survival statistics, to predict a probability of survival for a new patient as a function of time from a diagnosis (e.g., of an advanced stage cancer). The prediction can be provided to the new patient to, for example, improve their ability to plan for the future. This has the potential to improve the patient's quality of life. Alternatively or additionally, the system determines the cumulative survival probability with respect to time to generate a Kaplan-Meier (K-M) plot. The K-M plot can also be displayed. Additionally, or alternatively, the system can output the attributes and the medical outcomes of the cohort of patients. This may help to facilitate a clinical decision for a particular patient. For example, the system can output a summary of the attributes of the cohort of patients, along with a comparison of the attributes of the patients in the cohort. Such outputs allow a clinician to investigate the relevant attributes and to determine courses of actions (e.g., treatments) to improve the probability of survival of the patient.

In some examples, the system generates and displays multiple merged graphs. For example, the merged graph generated at step 710 is a first merged graph and the graphs are first graphs. The patient pathways of the patients are represented by second graphs (e.g., for a different group of patients). The system generates a second merged graph representing the patient pathways of the patients based on merging nodes of the second graphs representing a same type of diagnosis event, a same type of treatment event, or a same type of clinical outcome event into a merged node, and merging the edges between two merged nodes into a single edge. The system displays the second merged graph in the interface. For example, the second merged graph is displayed in the interface prior to displaying the first merged graph.

In some examples, the system can accept user input to retrieve and display additional information by interacting with a merged graph. For example, each of the patients is associated with a patient identifier in the database. Each merged node representing the same type of treatment event in the merged graph is associated with the patient identifier of each patient who has the event represented by the merged node in the patient's medical history data. The system receives, via the interface, a selection of a merged node representing a type of treatment event. The system determines patient identifiers associated with the merged node. The system retrieves, using the patient identifiers, medical history data of a subset of patients from the database. The system processes the medical history data of the subset of patients to generate a processing result and displays the processing result in the interface. The processing result may, for example, include a survival curve of the subset of patients. This can be repeated responsive to receiving selection of additional merged nodes (e.g., upon detecting a selection of a second merged node representing a second type of treatment event, the system repeats the process using second patient identifiers to generate a second processing result).

In some examples, each merged node representing a same type of treatment event or a same type of clinical outcome event in the merged graph is associated with the patient identifier of each patient who has the event represented by the merged node in the patient's medical history data, and a time of the event from when the patient is first diagnosed with a disease. The system displays a progression of events for each patient represented in the merged graph with respect to time based on the patient identifiers and the time of the event associated with each merged node representing a same type of treatment event or a same type of clinical outcome event in the merged graph.

The techniques described herein provide improved visualizations which can help a clinician to make sense of a great deal of data efficiently. For example, using the spaghetti-like patient pathways shown in FIG. 6A, a clinician would have to spend a great deal of time and energy to, if even possible, make sense of all the data shown and discern useful information. In contrast, using the merged graph shown in FIG. 6G, a clinician can clearly see the different pathways patients are taking, in an organized fashion. Further, the user interface allows the clinician to drill down into different categories of events (e.g., treatment events, biomarkers, etc.), which further aids in the interpretability of the merged graph.

In some aspects, the user interface also provides the advantage of allowing the user to interact with the merged graph to drill down into data for different patients or cohorts of patients. And, the system can compute and display additional metrics such as a survivability probability, time, and cost. This can be used to make informed decisions to investigate the relevant attributes and to determine courses of actions (e.g., treatments) to improve the probability of survival of the patient and/or improve the patient's ability to plan for the future and improve the patient's quality of life. Additional advantages include the ability to identify potential deviations in clinical care from clinical guidelines and potential inefficiencies in clinical workflow and resource management by navigating the interface to view the additional metrics.

### V. Computer System

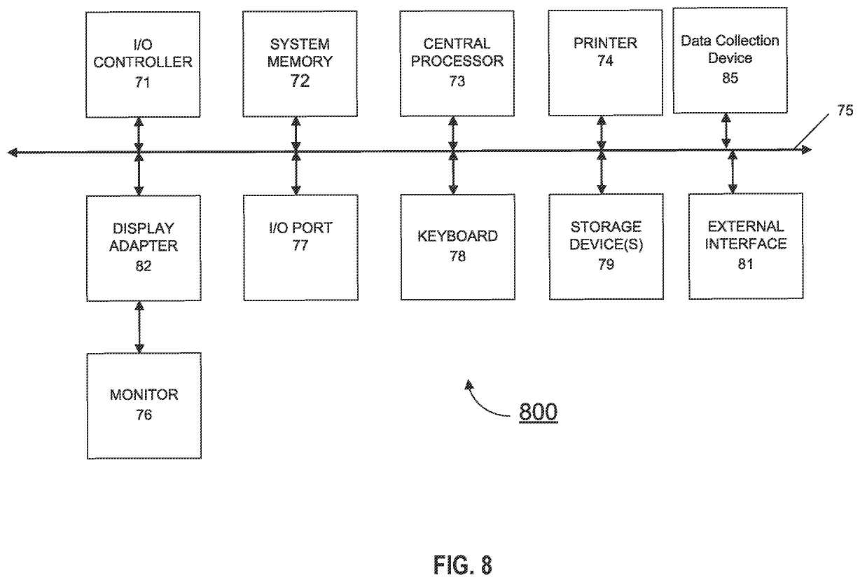

Any of the computer systems mentioned herein may utilize any suitable number of subsystems. Examples of such subsystems are shown in FIG. 8 in computer system 800. In some embodiments, a computer system includes a single computer apparatus, where the subsystems can be the components of the computer apparatus. In other embodiments, a computer system can include multiple computer apparatuses, each being a subsystem, with internal components. A computer system can include desktop and laptop computers, tablets, mobile phones and other mobile devices. In some embodiments, a cloud infrastructure (e.g., Amazon Web Services), a graphical processing unit (GPU), etc., can be used to implement the disclosed techniques.

The subsystems shown in FIG. 8 are interconnected via a system bus 75. Additional subsystems such as a printer 74, keyboard 78, storage device(s) 79, monitor 76, which is coupled to display adapter 82, and others are shown. Peripherals and input/output (I/O) devices, which couple to I/O controller 71, can be connected to the computer system by any number of means known in the art such as input/output (I/O) port 77 (e.g., USB, FireWire®). For example, I/O port 77 or external interface 81 (e.g. Ethernet, Wi-Fi, etc.) can be used to connect computer system 10 to a wide area network such as the Internet, a mouse input device, or a scanner. The interconnection via system bus 75 allows the central processor 73 to communicate with each subsystem and to control the execution of a plurality of instructions from system memory 72 or the storage device(s) 79 (e.g., a fixed disk, such as a hard drive, or optical disk), as well as the exchange of information between subsystems. The system memory 72 and/or the storage device(s) 79 may embody a computer readable medium. Another subsystem is a data collection device 85, such as a camera, microphone, accelerometer, and the like. Any of the data mentioned herein can be output from one component to another component and can be output to the user.

A computer system can include a plurality of the same components or subsystems, e.g., connected together by external interface 81 or by an internal interface. In some embodiments, computer systems, subsystem, or apparatuses can communicate over a network. In such instances, one computer can be considered a client and another computer a server, where each can be part of a same computer system. A client and a server can each include multiple systems, subsystems, or components.

Aspects of embodiments can be implemented in the form of control logic using hardware (e.g. an application specific integrated circuit or field programmable gate array) and/or using computer software with a generally programmable processor in a modular or integrated manner. As used herein, a processor includes a single-core processor, multi-core processor on a same integrated chip, or multiple processing units on a single circuit board or networked. Based on the disclosure and teachings provided herein, a person of ordinary skill in the art will know and appreciate other ways and/or methods to implement embodiments of the present invention using hardware and a combination of hardware and software.

Any of the software components or functions described in this application may be implemented as software code to be executed by a processor using any suitable computer language such as, for example, Java, C, C++, C #, Objective-C, Swift, or scripting language such as Perl or Python using, for example, conventional or object-oriented techniques. The software code may be stored as a series of instructions or commands on a computer readable medium for storage and/or transmission. A suitable non-transitory computer readable medium can include random access memory (RAM), a read only memory (ROM), a magnetic medium such as a hard-drive or a floppy disk, or an optical medium such as a compact disk (CD) or DVD (digital versatile disk), flash memory, and the like. The computer readable medium may be any combination of such storage or transmission devices.

Such programs may also be encoded and transmitted using carrier signals adapted for transmission via wired, optical, and/or wireless networks conforming to a variety of protocols, including the Internet. As such, a computer readable medium may be created using a data signal encoded with such programs. Computer readable media encoded with the program code may be packaged with a compatible device or provided separately from other devices (e.g., via Internet download). Any such computer readable medium may reside on or within a single computer product (e.g. a hard drive, a CD, or an entire computer system), and may be present on or within different computer products within a system or network. A computer system may include a monitor, printer, or other suitable display for providing any of the results mentioned herein to a user.

Any of the methods described herein may be totally or partially performed with a computer system including one or more processors, which can be configured to perform the steps. Thus, embodiments can be directed to computer systems configured to perform the steps of any of the methods described herein, potentially with different components performing a respective steps or a respective group of steps. Although presented as numbered steps, steps of methods herein can be performed at a same time or in a different order. Additionally, portions of these steps may be used with portions of other steps from other methods. Also, all or portions of a step may be optional. Additionally, any of the steps of any of the methods can be performed with modules, units, circuits, or other means for performing these steps.

The specific details of particular embodiments may be combined in any suitable manner without departing from the spirit and scope of embodiments of the invention. However, other embodiments of the invention may be directed to specific embodiments relating to each individual aspect, or specific combinations of these individual aspects.

The above description of example embodiments of the invention has been presented for the purposes of illustration and description. It is not intended to be exhaustive or to limit the invention to the precise form described, and many modifications and variations are possible in light of the teaching above.

A recitation of “a”, “an” or “the” is intended to mean “one or more” unless specifically indicated to the contrary. The use of “or” is intended to mean an “inclusive or,” and not an “exclusive or” unless specifically indicated to the contrary. Reference to a “first” component does not necessarily require that a second component be provided. Moreover reference to a “first” or a “second” component does not limit the referenced component to a particular location unless expressly stated.

All patents, patent applications, publications, and descriptions mentioned herein are incorporated by reference in their entirety for all purposes. None is admitted to be prior art.

1. A computer-implemented method, comprising:
obtaining, from a database, medical history data of patients, the medical history data including a history of one or more diagnosis events, one or more treatment events, and a clinical outcome event for each patient of the patients;
generating, for each patient and based on the medical history data, a patient pathway comprising a graph comprising nodes and edges connecting the nodes, the nodes representing the one or more diagnosis events, the one or more treatment events, and the clinical outcome event, and the edges representing temporal relations among the events represented by the nodes;
receiving, via an interface, one or more criteria to select a subset of the patient pathways of the patients;
selecting, based on the one or more criteria, graphs representing the subset of the patient pathways;
aggregating the subset of the patient pathways into a merged graph by:
identifying, among the subset of the patient pathways, a plurality of sets of nodes of the graphs, each set of nodes representing a same type of diagnosis event, a same type of treatment event, or a same type of clinical outcome event,
merging each set of the plurality of sets of nodes of the graphs, and
merging the edges between two merged nodes into a single edge; and
displaying the merged graph in the interface.

2. The method of claim 1, wherein a type of diagnosis event is based on a disease a patient is diagnosed with in an event.

3. The method of claim 1, wherein a type of treatment event is based on at least one of: a therapy administered to a patient, or a surgical operation administered to the patient.

4. The method of claim 1, wherein the type of clinical outcome event is based on one of: survival of a patient at a pre-determined time after the one or more treatment events, or death of the patient at the pre-determined time after the one or more treatment events.

5. The method of claim 1, wherein nodes representing the same type of diagnosis event, the same type of treatment event, or the same type of clinical outcome event represent events occurring at different times.

6. The method of claim 1, further comprising:
identifying one or more common patient pathways shared by a number of patients of the patients; and
associating each common patient pathway with a count of the number of patients sharing the common patient pathway;
ranking each patient pathway based on the associated count; and
selecting a number of top-ranked patient pathways as the subset of the patient pathways,
wherein the one or more criteria specify the number.

7. The method of claim 1, further comprising:
identifying one or more common types of treatment event shared by a number of patients of the patients;
associating each common type of treatment event with a percentage of the patients sharing the common type of treatment event; and
selecting the subset of the patient pathways including one or more common types of treatment event associated with a threshold percentage of the patients,
wherein the one or more criteria specify the threshold percentage.

8. The method of claim 1, further comprising:
identifying one or more common types of treatment event shared by a number of the patients;
associating each common type of treatment event with a subset of the patients sharing the common type of treatment event; and
selecting a first common type of treatment event and a second common type of treatment event to be included in the subset of the patient pathways,
wherein the first common type of treatment event is shared by a first subset of the patients,
wherein the second common type of treatment event is shared by a second subset of the patients who also have the first common type of treatment event prior to having the second common type of treatment event, and
wherein the first common type of treatment event and the second common type of treatment event are selected to be included in the subset of the patient pathways based on a percentage between the second subset and the first subset exceeding a threshold percentage.

9. The method of claim 1, further comprising:
clustering the patients based on a sequence of events in the patient pathway for each patient to produce a plurality of patient clusters,
wherein the selecting the graphs representing the subset of the patient pathways is further based on the patient clusters.

10. The method of claim 1, further comprising:
grouping the events into categories, wherein the merged graph is further based on the categories.

11. The method of claim 1, further comprising:
determining one or more metrics for each patient pathway of the subset of the patient pathways; and
displaying, in the interface, the one or more metrics concurrently with the merged graph.

12. The method of claim 11, wherein the one or more metrics of a patient pathway include at least one of: a number of patients who share the patient pathway, a total cost of one or more diagnosis events and one or more treatment events of the patient pathway, or a total time between a first diagnosis event and a clinical outcome event in the patient pathway.

13. The method of claim 1, wherein the merged graph is a first merged graph;
wherein the graphs are first graphs,
wherein the patient pathways of the patients are represented by second graphs,
wherein the method further comprises:
generating a second merged graph representing the patient pathways of the patients based on merging nodes of the second graphs representing a same type of diagnosis event, a same type of treatment event, or a same type of clinical outcome event into a merged node, and merging the edges between two merged nodes into a single edge; and
displaying the second merged graph in the interface.

14. The method of claim 13, wherein the second merged graph is displayed in the interface prior to displaying the first merged graph.

15. The method of claim 1,
wherein each of the patients is associated with a patient identifier in the database;
wherein each merged node representing a same type of treatment event in the merged graph is associated with the patient identifier of each patient who has the event represented by the merged node in the patient's medical history data; and
wherein the method further comprises:
receiving, via the interface, a selection of a first merged node representing a first type of treatment event;
determining first patient identifiers associated with the first merged node;
retrieving, using the first patient identifiers, medical history data of a first subset of patients from the database;
processing the medical history data of the first subset of patients to generate a first processing result; and
displaying the first processing result in the interface.

16. The method of claim 15, wherein the first processing result comprises a survival curve of the first subset of patients.

17. The method of claim 15, wherein the method further comprises:
receiving, via the interface, a selection of a second merged node representing a second type of treatment event;
determining second patient identifiers associated with the second merged node;
retrieving, using the second patient identifiers, medical history data of a second subset of patients from the database;
processing the medical history data of the first subset of patients to generate a second processing result; and
displaying the first processing result and the second processing result in the interface to provide a comparison between the first subset of patients and the second subset of patients.

18. The method of claim 15, wherein each merged node representing a same type of treatment event or a same type of clinical outcome event in the merged graph is associated with the patient identifier of each patient who has the event represented by the merged node in the patient's medical history data, and a time of the event from when the patient is first diagnosed with a disease; and
wherein the method further comprises displaying a progression of events for each patient represented in the merged graph with respect to time based on the patient identifiers and the time of the event associated with each merged node representing a same type of treatment event or a same type of clinical outcome event in the merged graph.

19. The method of claim 15, further comprising:
receiving medical data of a patient;
comparing the medical data of the patient with medical data of the first subset of patients; and
performing a clinical prediction for the patient based on the first processing result and a result of the comparison.

20. A computer system comprising:
one or more processors; and
a computer readable medium storing a plurality of instructions executable by the one or more processors for controlling the computer system to perform the following operations:
obtaining, from a database, medical history data of patients, the medical history data including a history of one or more diagnosis events, one or more treatment events, and a clinical outcome event for each patient of the patients;
generating, for each patient and based on the medical history data, a patient pathway comprising a graph comprising nodes and edges connecting the nodes, the nodes representing the one or more diagnosis events, the one or more treatment events, and the clinical outcome event, and the edges representing temporal relations among the events represented by the nodes;
receiving, via an interface, one or more criteria to select a subset of the patient pathways of the patients;
selecting, based on the one or more criteria, graphs representing the subset of the patient pathways;
aggregating the subset of the patient pathways into a merged graph by:
identifying, among the subset of the patient pathways, a plurality of sets of nodes of the graphs, each set of nodes representing a same type of diagnosis event, a same type of treatment event, or a same type of clinical outcome event,
merging each set of the plurality of sets of nodes of the graphs, and
merging the edges between two merged nodes into a single edge; and
displaying the merged graph in the interface.

21-24. (canceled)
